(19) United States
(12) Patent Application Publication
(10) Pub. No.: US 2016/0376610 A1
(43) Pub. Date: Dec. 29, 2016

(54) CELL CYCLE DEPENDENT GENOME REGULATION AND MODIFICATION

(71) Applicant: SIGMA-ALDRICH CO. LLC, ST. LOUIS, MO (US)

(72) Inventors: Gregory D. Davis, St. Louis, MO (US); Qingzhou Ji, St. Louis, MO (US); Carol A. Kreader, St. Louis, MO (US)

(21) Appl. No.: 15/192,095

(22) Filed: Jun. 24, 2016

Related U.S. Application Data
(60) Provisional application No. 62/184,131, filed on Jun. 24, 2015.

(51) Int. Cl. C12N 15/90; C07K 14/435; C07K 14/47; C12N 9/22

(52) U.S. Cl. CPC C12N 15/907; C12N 9/22; C07K 14/43595; C07K 14/4703; C07K 2319/60; C07K 2319/00

(57) ABSTRACT

Fusion protein comprising a programmable DNA modification protein and a cell cycle regulated protein, and methods of using the fusion protein to modify chromosomal sequences and/or regulate gene expression in a cell cycle dependent manner.

### CROSS-REFERENCE TO RELATED APPLICATIONS

This application claims the benefit of priority to U.S. Provisional Application Ser. No. 62/184,131, filed Jun. 24, 2015, the disclosure of which is hereby incorporated by reference in its entirety.

### FIELD

Compositions and methods for modifying chromosomal sequences or regulating expression of chromosomal sequences in a cell cycle dependent manner.

### BACKGROUND

Programmable endonucleases have increasingly become an important tools for targeted genome engineering or modification in eukaryotes. Programmable endonucleases such as RNA-guided clustered regularly interspersed short palindromic repeats (CRISPR)/CRISPR-associated (Cas) (CRISPR/Cas) nucleases, zinc finger nucleases (ZFNs), and transcription activator-like effector nucleases (TALENs) are engineered to target a specific chromosomal sequence and introduce a double stranded break at a target site. The double stranded break can be repaired by homology directed repair (HDR) processes or non-homologous end joining (NHEJ) processes. However, the ratio of HDR to NHEJ is low in particular mammalian and plant cell types and it is established that HDR components are activated during specific phases of the cell cycle (Maynahan et al., Nature Rev. Mol. Cell Biol., 2010, 11(3):196-207).

Thus, there is a need for means for restricting expression of targeted endonucleases to specific phases of the cell cycle. For example, if a targeting endonuclease is expressed only during the S/G2 phases of the cell cycle, the ratio of HDR to NHEJ may increase significantly. A possible secondary benefit of cell cycle regulated expression of targeting endonucleases is a reduction in off-target NHEJ-mediated errors in genome editing processes that require HDR to achieve the desired outcome. Thus, by reducing expression of the targeting endonuclease during the M/G1 phases, a significant fraction of opportunities for off-target nuclease activity will be reduced in each cell in a population, and previous studies have shown the reductions in the duration of targeted nuclease expression can elevate on-target to off-target ratios (Kim et al., Genome Res., 2014, 24(6):1012-1019).

### SUMMARY

Among the various aspects of the present disclosure is the provision of a fusion protein comprising a programmable DNA modification protein and a cell cycle regulated protein. In some embodiments, the programmable DNA modification protein has nuclease activity, and it is chosen from a CRISPR/Cas nuclease, a CRISPR/Cas nickase, a DNA-guided Argonaute endonuclease, a zinc finger nuclease, a transcription activator-like effector nuclease, a meganuclease, or a chimeric protein comprising a programmable DNA-binding domain and a nuclease domain. In some aspects, the CRISPR/Cas nuclease or nickase further comprises a guide RNA, and the DNA-guided Argonaute endonuclease further comprises a single-stranded guide DNA. In other embodiments, the programmable DNA modification protein has non-nuclease activity, wherein it is a chimeric protein comprising a programmable DNA-binding domain and a non-nuclease modification domain. The programmable DNA-binding domain can be chosen from a CRISPR/Cas nuclease modified to lack all nuclease activity, a DNA-guided Argonaute endonuclease modified to lack all nuclease activity, a meganuclease modified to lack all nuclease activity, a zinc finger protein, or a transcription activator-like effector; and the non-nuclease domain can be chosen from a transcriptional activation domain, a transcriptional repressor domain, a histone acetyltransferase domain, a histone deacetylase domain, a histone methyltransferase domain, a histone demethylase domain, a DNA methyltransferase domain, or a DNA demethylase domain. In certain embodiments, the cell cycle regulated protein is chosen from geminin, cyclin A, cyclin B, cyclin D, CDC20, or securin. In various embodiments, the fusion protein further comprises at least one nuclear localization signal, at least one cell-penetrating domain, at least one marker domain, and/or at least one linker. In one embodiment, the programmable DNA modification protein is a Cas9 nuclease or derivative thereof and the cell cycle regulated protein is geminin. In another embodiment, the fusion protein comprises SEQ ID NO:14.

Another aspect of the present disclosure encompasses a nucleic acid encoding the above-described fusion protein. In some embodiments, the nucleic acid encoding the fusion protein is operably linked to an expression control sequence. In certain embodiments, the expression control sequence is a constitutive promoter sequence, a cell cycle regulated promoter sequence, a derivative, or fragment thereof. In other embodiments, the expression control sequence is a 3′ untranslated region that is targeted by one or more cell cycle regulated microRNAs, or the expression control sequence codes a reverse complement of a cell cycle regulated microRNA. In still other embodiments, the nucleic acid encoding the fusion protein is codon optimized for translation in a eukaryotic cell. In still other embodiments, the nucleic acid encoding the fusion protein is part of a vector.

A further aspect of the present disclosure provides cells comprising the above-described fusion protein or the above-described nucleic acid. In some embodiments, the nucleic acid is extrachromosomal. In other embodiments, the nucleic acid is integrated into a chromosome. In various embodiments, the cell is a human cell, a non-human mammalian cell, a non-mammalian vertebrate cell, a stem cell, a non-human one cell embryo, an invertebrate cell, a plant cell, or a single cell eukaryotic organism. In some embodiments, the fusion protein is degraded during M phase and/or during the transition from M phase to G1 phase of the cell cycle.

Another aspect of the present disclosure encompasses methods for modifying chromosomal sequences and/or regulating expression of chromosomal sequences in a cell cycle dependent manner. One method comprises introducing into the cell a nucleic acid encoding the above-described fusion protein, and optionally a donor polynucleotide comprising at least one sequence having substantial sequence identity with a target site in the chromosomal sequence. The fusion protein is expressed in a portion of the, such that the fusion protein modifies the chromosomal sequence and/or regulates expression of the chromosomal sequence during that portion of the cell cycle. In embodiments in which the programmable DNA modification protein of the fusion protein is a targeting endonuclease that introduces a double stranded break at a target site in the chromosomal sequence, repair of the double-stranded break has a ratio of homology directed repair (HDR) to non-homologous end joining (NHEJ) that is increased relative to a corresponding targeting endonuclease that is not fused to a cell cycle regulated protein.

Other aspects and iterations of the disclosure are detailed below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 presents a map of an expression vector encoding a Cas9-NLS-GFP-geminin fusion protein. tEF1a=truncated human elongation factor-1 promoter alpha; WPRE=woodchuck hepatitis virus posttranscriptional regulatory element; LTR=long terminal repeat.

FIG. 2A presents fluorescence images (top) and differential contrast images (bottom) at the indicated time points of U2OS cells expressing Cas9-GFP-Gemimin fusion protein.

FIG. 2B illustrates the phases of the cell cycle in which Cas9-GFP-Gemimin fusion protein (indicated by the thicker arrow) is expressed

FIG. 3A presents the results of a Cel-1 nuclease assay in U2OS cells. Lane 1, DNA markers. Lane 2, cells transfected with Cas9-GFP-Gem plasmid only. Lane 3, cells transfected with Cas9-GFP-Gem plasmid+AAVS1-gRNA. Lane 4, cells transfected with Cas9-GFP-Gem plasmid+AAVS1-gRNA+AAVS1-ssODN. Lane 5, cells transfected with Cas9 plasmid only. Lane 6, cells transfected with Cas9 plasmid+AAVA1-gRNA. Lane 7, cells transfected with Cas9 plasmid+AAVS1-gRNA+AAVS1 ss-ODN.

FIG. 3B shows the results of a RFLP assay in U2OS cells. Lane 1, DNA markers. Lane 2, cells transfected with Cas9-GFP-Gem plasmid only. Lane 3, cells transfected with Cas9-GFP-Gem plasmid+AAVS1-gRNA. Lane 4, cells transfected with Cas9-GFP-Gem plasmid+AAVS1-gRNA+AAVS1-ssODN. Lane 5, cells transfected with Cas9 plasmid only. Lane 6, cells transfected with Cas9 plasmid+AAVA1-gRNA. Lane 7, cells transfected with Cas9 plasmid+AAVS1-gRNA+AAVS1 ss-ODN.

FIG. 4 illustrates that Cas9-GFP-Geminin increased HDR/NHEJ ratio in K562 cells. Plotted is the relative ratio of HDR to NHEJ of Cas9 (ratio set to 1) and Cas9-GFP-Geminin.

### DETAILED DESCRIPTION

The present disclosure provides compositions and methods for targeting specific chromosomal sequences for genome modification or regulation during particular phases of the cell cycle. Provided herein are (i) fusion proteins comprising programmable DNA modification proteins linked to cell cycle regulated proteins, (ii) nucleic acids encoding the fusion proteins, (iii) cells comprising the above-mentioned nucleic acids, wherein the cells express fusion proteins whose levels fluctuate during the cell cycle, and (iv) methods of using the fusion proteins to target specific chromosomal sequences and mediate genome modification or regulation during specific phases of the cell cycle.

### (I) Fusion Proteins

One aspect of the present disclosure provides fusion proteins comprising a programmable DNA modification protein and a cell cycle regulated protein. A programmable DNA modification protein is a protein that binds to a specific target sequence in a chromosome and modifies the DNA or a protein associated with the DNA at or near the target sequence. Thus, a programmable DNA modification protein comprises a DNA-binding domain and a modification domain. The DNA-binding domain is programmable, meaning that it can be designed or engineered to recognize and bind different DNA sequences. A cell cycle regulated protein is a protein whose levels fluctuate during the cell cycle. For example, the synthesis and/or degradation of a cell cycle regulated protein is regulated in a cell cycle dependent manner. Thus, the level of a fusion protein comprising a cell cycle regulated protein can also fluctuate during the cell cycle.

The programmable DNA modification protein can be linked to the amino terminus or the carboxyl terminus of the cell cycle regulated protein, thereby forming the fusion protein. The fusion proteins disclosed herein can further comprise additional domains, such as one or more nuclear localization signals, one or more cell-penetrating domains, or one or more marker domains, and/or one or more linkers.

### (a) Programmable DNA Modification Proteins

The programmable DNA modification protein of the fusion proteins disclosed herein comprises a programmable DNA-binding domain and a modification domain.

The programmable DNA-binding domain can be designed or engineered to recognize and bind different DNA sequences. In some embodiments, the DNA binding is mediated by interaction between the protein and the target DNA. Thus, the DNA-binding domain can be programmed to bind a DNA sequence of interest by protein engineering. In other embodiments, DNA-binding is mediated by a guide nucleic acid that interacts with the protein and the target DNA. In such instances, the programmable DNA-binding domain can be targeted to a DNA sequence of interest by designing the appropriate guide nucleic acid.

In some embodiments, the programmable DNA modification protein comprises a nuclease modification domain and, thus, has nuclease activity. Thus, the programmable DNA modification protein is a targeting endonuclease that cleaves DNA at a targeted site. The cleavage can be double-stranded or single-stranded. The cleavage can be repaired by homology directed repair (HDR) or non-homologous end-joining (NHEJ) repair processes. Examples of programmable DNA modification proteins comprising nuclease domains (or targeting endonucleases) include, without knit, CRISPR/Cas nucleases, CRISPR/Cas nickases, DNA-guided Argonaute endonucleases, zinc finger nucleases, transcription activator-like effector nucleases, meganucleases, or chimeric proteins comprising a programmable DNA-binding domain and a nuclease domain. Programmable DNA modification proteins having nuclease activity are detailed below in sections (I)(a)(i)-(vii).

In other embodiments, the programmable DNA modification protein comprises a non-nuclease modification domain (e.g., transcriptional regulation domain, histone acetylation domain, etc.) such that the programmable DNA modification protein modifies the structure and/or activity of the DNA and/or protein(s) associated with the DNA. Thus, the programmable DNA modification protein is a chimeric protein comprising a programmable DNA-binding domain and a non-nuclease domain. Such proteins are detailed below in section (I)(a)(viii).

The programmable DNA modification proteins can comprise wild-type or naturally-occurring DNA-binding and/or modification domains, modified versions of naturally-occurring DNA-binding and/or modification domains, synthetic or artificial DNA-binding and/or modification domains, or combinations thereof.

(i) CRISPR/Cas Nucleases

In some embodiments, the programmable DNA modification protein having nuclease activity can be a RNA-guided CRISPR/Cas nuclease. The CRISPR/Cas is guided by a guide RNA to a target sequence at which it introduces a double-stranded break in the DNA.

The CRISPR/Cas nuclease can be derived from a type I (i.e., IA, IB, IC, ID, IE, or IF), type II (i.e., IIA, IIB, or IIC), type III (i., IIIA or IIIB), or type V CRISPR system, which are present in various bacteria and archaea. The CRISPR/Cas system can be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR proteins include Cas proteins, Cpf proteins, Cmr proteins, Csa proteins, Csb proteins, Csc proteins, Cse proteins, Csf proteins, Csm proteins, Csn proteins, Csx proteins, Csy proteins, Csz proteins, and derivatives or variants thereof. In specific embodiments, the CRIPSR/Cas nuclease can be a type II Cas9 protein, a type V Cpf1 protein, or a derivative thereof. In some embodiments, the CRISPR/Cas nuclease can be Streptococcus pyogenes Cas9 (SpCas9) or Streptococcus thermophilus Cas9 (StCas9). In other embodiments, the CRISPR/Cas nuclease can be Campylobacter jejuni Cas9 (CjCas9). In alternate embodiments, the CRISPR/Cas nuclease can be Francisella novicida Cas9 (FnCas9). In yet other embodiments, the CRISPR/Cas nuclease can be Francisella novicida Cpf1 (FnCpf1).

In general, the CRISPR/Cas nuclease comprises a RNA recognition and/or RNA binding domain, which interacts with the guide RNA. The CRISPR/Cas nuclease also comprises at least one nuclease domain having endonuclease activity. For example, a Cas9 protein can comprise a RuvC-like nuclease domain and a HNH-like nuclease domain, and a Cpf1 protein can comprise a RuvC-like domain. CRISPR/Cas nucleases can also comprise DNA binding domains, helicase domains, RNase domains, protein-protein interaction domains, dimerization domains, as well as other domains.

The CRISPR/Cas nuclease can be associated with a guide RNA (gRNA). The guide RNA interacts with the CRISPR/Cas nuclease to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY and PAM sequences for Cpf1 include 5′-TTN (wherein N is defined as any nucleotide, W is defined as either A or T, and Y is defined an either C or T). Each gRNA comprises a sequence that is complementary to the target sequence (e.g., a Cas9 gRNA can comprise GN₁₇₋₂₀GG). The gRNA can also comprise a scaffold sequence that forms a stem loop structure and a single-stranded region. The scaffold region can be the same in every gRNA. In some embodiments, the gRNA can be a single molecule (i.e., sgRNA). In other embodiments, the gRNA can be two separate molecules. Those skilled in the art are familiar with gRNA design and construction, e.g., gRNA design tools are available on the internet or from commercial sources.

(ii) CRISPR/Cas Nickases

In other embodiments, the programmable DNA modification protein having nuclease activity can be a CRISPR/Cas nickase. CRISPR/Cas nickases are similar to the CRISPR/Cas nucleases described above except that the CRISPR/Cas nuclease is modified to cleave only one strand of DNA. Thus, a single CRISPR/Cas nickase in combination with a guide RNA can create a single-stranded break or nick in the DNA. Alternatively, a CRISPR/Cas nickase in combination with a pair of offset gRNAs can create a double-stranded break in the DNA.

A CRISPR/Cas nuclease can be converted to a nickase by one or more mutations and/or deletions. For example, a Cas9 nickase can comprise one or more mutations in one of the nuclease domains, wherein the one or more mutations can be D10A, E762A, and/or D986A in the RuvC-like domain or the one or more mutations can be H840A (or H839A), N854A and/or N863A in the HNH-like domain.

(iii) ssDNA-Guided Argonaute Endonucleases

In alternate embodiments, the programmable DNA modification protein having nuclease activity can be a single-stranded DNA-guided Argonaute endonuclease. Argonautes (Agos) are a family of endonucleases the use 5′-phosphorylated short single-stranded nucleic acids as guides to cleave nucleic acid targets. Some prokaryotic Agos use single-stranded guide DNAs and create double-stranded breaks in DNA (Gao et al., Nature Biotechnology, 2016, May 2. doi: 10.1038/nbt.3547). The ssDNA-guided Ago endonuclease can be associated with a single-stranded guide DNA.

The Ago endonuclease can be derived from Alistipes sp., Aquifex sp., Archaeoglobus sp., Bacteroides sp., Bradyrhizobium sp., Burkholderia sp., Cellvibrio sp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. In some embodiments, the Ago endonuclease can be Natronobacterium gregoryi Ago (NgAgo). In other embodiments, the Ago endonuclease can be Thermus thermophilus Ago (TtAgo). In still further embodiments, the Ago endonuclease can be Pyrococcus furiosus (PfAgo).

The single-stranded guide DNA (gDNA) is complementary to the target site in the DNA. The target site has no sequence limitations and does not require a PAM. The gDNA generally ranges in length from about 15-30 nucleotides. In some embodiment, the gDNA can be about 24 nucleotides in length. The gDNA may comprise a 5′ phosphate group. Those skilled in the art are familiar with ssDNA oligonucleotide design and construction.

(iv) Zinc Finger Nucleases

In still other embodiments, the programmable DNA modification protein having nuclease activity can be a zinc finger nuclease (ZFN). A ZFN comprise a DNA-binding zinc finger region and a nuclease domain. The zinc finger region can comprise from about two to seven zinc fingers, for example, about four to six zinc fingers, wherein each zinc finger binds three nucleotides. The zinc finger region can be engineered to recognize and bind to any DNA sequence. Zinc finger design tools or algorithms are available on the internet or from commercial sources. The zinc fingers can be linked together using suitable linker sequences.

A ZFN also comprises a nuclease domain, which can be obtained from any endonuclease or exonuclease. Non-limiting examples of endonucleases from which a nuclease domain can be derived include, but are not limited to, restriction endonucleases and homing endonucleases. In some embodiments, the nuclease domain can be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MboII, and SapI. In some embodiments, the nuclease domain can be a FokI nuclease domain or a derivative thereof. The type II-S nuclease domain can be modified to facilitate dimerization of two different nuclease domains. For example, the cleavage domain of FokI can be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI nuclease domains are targets for modification. For example, one modified FokI domain can comprise Q486E, I499L, and/or N496D mutations, and the other modified FokI domain can comprise E490K, I538K, and/or H537R mutations.

(v) Transcription Activator-Like Effector Nucleases

In alternate embodiments, the programmable DNA modification protein having nuclease activity can be a transcription activator-like effector nuclease (TALEN). TALENs comprise a DNA-binding domain composed of highly conserved repeats derived from transcription activator-like effectors (TALEs) that is linked to a nuclease domain. TALEs are proteins secreted by plant pathogen Xanthomonas to alter transcription of genes in host plant cells. TALE repeat arrays can be engineered via modular protein design to target any DNA sequence of interest. The nuclease domain of TALENs can be any nuclease domain as described above in section (I)(a)(iv). In specific embodiments, the nuclease domain is derived from FokI (Sanjana et al., 2012, Nat Protoc, 7(1):171-192).

(vi) Meganucleases or Rare-Cutting Endonucleases

In still other embodiments, the programmable DNA modification protein having nuclease activity can be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. In some embodiments, the meganuclease can be I-SceI or variants thereof. A meganuclease can be targeted to a specific chromosomal sequence by modifying its recognition sequence using techniques well known to those skilled in the art.

In alternate embodiments, the programmable DNA modification protein having nuclease activity can be a rare-cutting endonuclease or derivative thereof. Rare-cutting endonucleases are site-specific endonucleases whose recognition sequence occurs rarely in a genome, preferably only once in a genome. The rare-cutting endonuclease may recognize a 7-nucleotide sequence, an 8-nucleotide sequence, or longer recognition sequence. Non-limiting examples of rare-cutting endonucleases include NotI, AscI, PacI, AsiSI, SbfI, and FseI.

(vii) Chimeric Proteins Comprising Nuclease Domains

In yet additional embodiments, the programmable DNA modification protein having nuclease activity can be a chimeric protein comprising a nuclease domain and a programmable DNA-binding domain. The nuclease domain can be any of those described above in section (I)(a)(iv), a nuclease domain derived from a CRISPR/Cas nuclease (e.g., RuvC-like or HNH-like nuclease domains of Cas9 or nuclease domain of Cpf1), a nuclease domain derived from an Ago nuclease, or a nuclease domain derived from a meganuclease or rare-cutting endonuclease.

The programmable DNA-binding domain of the chimeric protein can be a programmable endonuclease (i.e., CRISPR/CAS nuclease, Ago nuclease, or meganuclease) modified to lack all nuclease activity. Alternatively, the programmable DNA-binding domain of the chimeric protein can be a programmable DNA-binding protein such as, e.g., a zinc finger protein or a TALE. In some embodiments, the programmable DNA-binding domain can be a catalytically inactive CRISPR/Cas nuclease in which the nuclease activity was eliminated by mutation and/or deletion. For example, the catalytically inactive CRISPR/Cas protein can be a catalytically inactive (dead) Cas9 (dCas9) in which the RuvC-like domain comprises a D10A, E762A, and/or D986A mutation and the HNH-like domain comprises a H840A (or H839A), N854A and/or N863A mutation. Alternatively, the catalytically inactive CRISPR/Cas protein can be a catalytically inactive (dead) Cpf1 protein comprising comparable mutations in the nuclease domain. In other embodiments, the programmable DNA-binding domain can be a catalytically inactive Ago endonuclease in which nuclease activity was eliminated by mutation and/or deletion. In still other embodiments, the programmable DNA-binding domain can be a catalytically inactive meganuclease in which nuclease activity was eliminated by mutation and/or deletion, e.g., the catalytically inactive meganuclease can comprise a C-terminal truncation.

(viii) Chimeric Proteins Comprising Non-Nuclease Domains

In alternate embodiments, the programmable DNA modification protein can be a fusion protein comprising a non-nuclease domain and a programmable DNA-binding domain. Suitable programmable DNA-binding domains are described above in section (I)(a)(vii). Examples of suitable non-nuclease domains include transcriptional regulation domains or epigenetic modification domains.

In some embodiments, the non-nuclease domain of the programmable DNA modification protein having non-nuclease activity can be a transcriptional regulation domain. A transcriptional regulation domain can be a transcriptional activation domain or a transcriptional repressor domain. In general, a transcriptional activation domain interacts with transcriptional control elements and/or transcriptional regulatory proteins (i.e., transcription factors, RNA polymerases, etc.) to increase and/or activate transcription of a gene, and a transcriptional repressor domain interact with said protein to decrease or repress transcription of a gene. Suitable transcriptional activation domains include, without limit, herpes simplex virus VP16 domain, VP64 (which is a tetrameric derivative of VP16), NFκB p65 activation domains, p53 activation domains 1 and 2, CREB (cAMP response element binding protein) activation domains, E2A activation domains, activation domain from human heat-shock factor 1 (HSF1), or NFAT (nuclear factor of activated T-cells) activation domains. Non-limiting examples of suitable transcriptional repressor domains include inducible cAMP early repressor (ICER) domains, Kruppel-associated box A (KRAB-A) repressor domains, YY1 glycine rich repressor domains, Sp1-like repressors, E(spl) repressors, IκB repressor, or MeCP2. Transcriptional activation or transcriptional repressor domains can be genetically fused to the DNA binding protein or bound via noncovalent protein-protein, protein-RNA, or protein-DNA interactions.

In other embodiments, the non-nuclease domain of the programmable DNA modification protein having non-nuclease activity can be an epigenetic modification domain. In general, epigenetic modification domains alter gene expression by modifying the histone structure and/or chromosomal structure. Suitable epigenetic modification domains include, without limit, histone acetyltransferase domains, histone deacetylase domains, histone methyltransferase domains, histone demethylase domains, DNA methyltransferase domains, and DNA demethylase domains.

### (b) Cell Cycle Regulated Proteins

The fusion protein also comprises a cell cycle regulated protein, derivative, or fragment thereof. A cell cycle regulated protein is a protein whose levels fluctuate during the cell cycle. Suitable cell cycle regulated proteins include those that are targeted for degradation during M phase and/or early G1 phase of the cell cycle. Non-limiting examples of suitable cell cycle regulated proteins include geminin, cyclin A (e.g., cyclin A1 or cyclin A2), cyclin B (e.g., cyclin B1, cyclin B2, or cyclin B3), cyclin D (e.g., cyclin D1, cyclin D2, or cyclin D3), CDC20 (cell division cycle 20), and securin. In specific embodiments, the cell cycle regulated protein is geminin (GenBank Accession number NP-056979), which is a DNA replication inhibitor (of about 25 kDa) that is expressed during S and G2 phases of the cell cycle and is degraded by the anaphase-promoting complex during the metaphase-anaphase transition.

### (c) Optional Additional Domains

The fusion protein can further comprise at least one nuclear localization signal, at least one cell-penetrating domain, at least one marker domain, and/or at least one linker.

In certain embodiments, the fusion protein can comprise at least one nuclear localization signal. In general, an NLS comprises a stretch of basic amino acids. Nuclear localization signals are known in the art (see, e.g., Lange et al., J. Biol. Chem., 2007, 282:5101-5105). For example, in one embodiment, the NLS can be a monopartite sequence, such as PKKKRKV (SEQ ID NO: 1) or PKKKRRV (SEQ ID NO: 2). In another embodiment, the NLS can be a bipartite sequence. In still another embodiment, the NLS can be KRPAATKKAGQAKKKK (SEQ ID NO: 3). The NLS can be located at the N-terminus, the C-terminal, or in an internal location of the fusion protein.

In other embodiments, the fusion protein can comprise at least one cell-penetrating domain. In one embodiment, the cell-penetrating domain can be a cell-penetrating peptide sequence derived from the HIV-1 TAT protein. As an example, the TAT cell-penetrating sequence can be GRKKRRQRRRPPQPKKKRKV (SEQ ID NO: 4). In another embodiment, the cell-penetrating domain can be TLM (PLSSIFSRIGDPPKKKRKV; SEQ ID NO: 5), a cell-penetrating peptide sequence derived from the human hepatitis B virus. In still another embodiment, the cell-penetrating domain can be MPG (GALFLGWLGAAGSTMGAPKKKRKV; SEQ ID NO: 6 or GALFLGFLGAAGSTMGAWSQPKKKRKV; SEQ ID NO: 7). In additional embodiments, the cell-penetrating domain can be Pep-1 (KETWWETWWTEWSQPKKKRKV; SEQ ID NO: 8), VP22, a cell penetrating peptide from Herpes simplex virus, or a polyarginine peptide sequence. The cell-penetrating domain can be located at the N-terminus, the C-terminal, or in an internal location of the fusion protein.

In still other embodiments, the fusion protein can comprise at least one marker domain. Non-limiting examples of marker domains include fluorescent proteins, purification tags, and epitope tags. In some embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1,), blue fluorescent proteins (e.g. EBFP, EBFP2, Azurite, mKalama1, GFPuv, Sapphire, T-sapphire,), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, 51, T7, V5, VSV-G, 6×His, biotin carboxyl carrier protein (BCCP), and calmodulin.

In some embodiments, the fusion protein can comprise at least one linker. For example, the programmable DNA modification protein, the cell cycle regulated protein, and other optional domains can be linked via one or more linkers. The linker can be flexible (e.g., comprising small, non-polar (e.g., Gly) or polar (e.g., Ser, Thr) amino acids). Non-limiting examples of flexible linkers include GGSGGGSG (SEQ ID NO:9), (GGGGS)₁₋₄ (SEQ ID NO:10), and (Gly)₆₋₈. Alternatively, the linker can be rigid, such as (EAAAK)₁₋₄ (SEQ ID NO:11), A(EAAAK)₂₋₅A (SEQ ID NO:12), PAPAP, (AP)₆₋₈, and (XP)ₙ, wherein X is any amino acid, but preferably Ala, Lys, or Glu. Examples of suitable linkers are well known in the art and programs to design linkers are readily available (Crasto et al., Protein Eng., 2000, 13(5):3096-312). In alternate embodiments, the programmable DNA modification protein, the cell cycle regulated protein, and other optional domains can be linked directly.

### (e) Specific Fusion Proteins

In specific embodiments, the programmable DNA modification protein of the fusion protein is a Cas9 protein (i.e., nuclease or nickase) and the cell cycle regulated protein is geminin. In other embodiments, the programmable DNA modification protein is a zinc finger nuclease (ZFN). The fusion protein can further comprise a nuclear localization signal (NLS) and/or a fluorescent protein (FP). Non-limiting examples of specific fusion proteins are presented below:

Specific fusion proteins (NH₂—COOH)

Cas9-geminin
geminin-Cas9
Cas9-NLS-geminin
Cas9-geminin-NLS
geminin-NLS-Cas9
geminin-Cas9-NLS
NLS-Cas9-geminin
NLS-geminin-Cas9
Cas9-NLS-FP-geminin
Cas9-NLS-geminin-FP
Cas9-geminin-FP-NLS
Cas9-geminin-NLS-FP
Cas9-FP-geminin-NLS
Cas9-FP-NLS-geminin
geminin-NLS-FP-Cas9
geminin-NLS-Cas9-FP
geminin-FP-NLS-Cas9
geminin-FP-Cas9-NLS
geminin-Cas9-NLS-FP
gGeminin-Cas9-FP-NLS
ZFN-geminin
ZFN-NLS-geminin
geminin-ZFN
geminin-NLS-ZFN
ZFN-geminin-FP
ZFN-FP-geminin
geminin-ZFN-FP
geminin-FP-ZFN
ZFN-NLS-geminin-FP
ZFN-NLS-FP-geminin
geminin-NLS-ZFN-FP
geminin-NLS-FP-ZFN

### (II) Nucleic Acids Encoding Fusion Proteins

Another aspect of the present disclosure provides nucleic acids encoding any of the fusion proteins described above in section (I). The nucleic acid encoding the fusion protein can be RNA or DNA. In one embodiment, the nucleic acid encoding the fusion protein is mRNA. In another embodiment, the nucleic acid encoding the fusion protein is DNA. The DNA encoding the fusion protein can be part of a vector (see below).

In some embodiments, the nucleic acid encoding the fusion protein can be operably linked to at least one sequence that regulates expression of the fusion protein in a eukaryotic cell. In certain embodiments, the nucleic acid encoding the fusion protein can be operably linked to a constitutive transcriptional control sequence. In other embodiments, the encoding nucleic acid can be operably linked to one or more sequences that permit cell cycle dependent expression of the fusion protein. Thus, the fusion protein coding sequence can be operably linked to a transcriptional control sequence, derivative, or fragment thereof that is regulated by (activating or repressive) transcription factors in a cell cycle dependent manner (Whitfield et al., Mol. Biol. Cell, 2002, 13:1977-2000) and/or a sequence that interacts with micro RNAs (miRNAs) in a cell cycle dependent manner (Bueno et al., Biochim. Biophys. Acta, 2011, 1812:592-601).

Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor-1 promoter alpha (e.g., truncated human elongation factor-1 promoter alpha), ubiquitin promoters, actin promoters, tubulin promoters, immunoglobulin promoters, derivatives thereof, fragments thereof, or combinations of any of the foregoing.

The cell cycle regulated promoter control sequence, derivative, or fragment thereof can be from a gene whose expression is regulated in a cell cycle dependent manner. For example, the promoter control sequence can be a consensus binding sequence for an activating transcription factor that is expressed or activated during G2 phase of the cell cycle, or conversely, a consensus binding sequence for a repressive transcription factor that is expressed or activated during G1 or S phases of the cell cycle. In some embodiments, the sequence encoding the fusion protein can be linked to a sequence that responds to G2 activating transcription factors and a sequence that responds to G1/S repressive transcription factors.

Non-limiting examples of genes expressed during G2 include TOP2A (topoisomerase II alpha), CDKN2C (cyclin-dependent kinase inhibitor 2C), CCNA2 (cyclin A2), CCNF (cyclin F), CDC2 (cell division cycle 2), CDC25C (cell division cycle 25C), CKS1 (cyclin-dependent kinases regulatory subunit 1), and GMNN (geminin). Examples of genes expressed during S phase include, without limit, BRCA1 (breast cancer type 1 susceptibility protein), CDC45L (cell division cycle 45-like), DHFR (dihydrofolate reductase), histones H1, H2A, H2B, H4, RRM1 (ribonucleotide reductase M1), RRM2 (ribonucleotide reductase M2), and TYMS (thymidylate synthetase). Non-limiting examples of genes expressed during G1/S include CCNE1 (cyclin E1), CCNE2 (cyclin E2), CDC25A (cell division cycle 25A), CDC6 (cell division cycle 6), E2F1 (E2F transcription factor 1), MCM2 (minichromosome maintenance complex component 2), MCM6 (minichromosome maintenance complex component 6), NPAT (nuclear protein, ataxia-telangiectasia locus), PCNA (proliferating cell nuclear antigen), SLBP (stem-loop binding protein), MSH2 (DNA mismatch repair protein), and NASP (nuclear autoantigenic sperm protein). Examples of genes expressed during G2/M include, but are not limited to, BIRC5 (baculoviral IAP repeat containing 5), BUB1 (mitotic checkpoint serine/threonine kinase), BUB1B (mitotic checkpoint serine/threonine kinase B), CCNB1 (cyclin B1), CCNB2 (cyclin B2), CENPA (centromere protein A), CENPF (centromere protein F), CDC20 (cell cycle dependent 20 protein), CDC25B (cell division cycle 25B), CDKN2D, p19 (cyclin-dependent kinase inhibitor 2D), CKS2 (cyclin-dependent kinases regulatory subunit 2), E2F5 (E2F Transcription Factor 5), PLK (Polo-like kinase), RACGAP1 (Rac GTPase-activating protein 1), RAB6KIFL (Rabkinesin-6/Rab6-KIFL/MKIp2), STK15 (serine/threonine kinase 15 or Aurora kinase), and STL6 (serine/threonine kinase 6 or Aurora kinase A).

Alternatively, the nucleic acid encoding the fusion protein can be operably linked to a sequence that interacts with miRNAs in a cell cycle dependent manner. For example, the cell cycle regulated sequence can be a 3′ untranslated region (3′-UTR) or fraction thereof of a gene whose expression is inhibited by miRNAs (i.e., by blocking translation and/or destabilizing the transcript) during particular phase(s) of the cell cycle. Gene transcripts whose expression is inhibited by miRNAs during G1 phase include cyclin D, cyclin E, CDC25A, CDK2, CDK4, and CDK6. Alternatively, the cell cycle regulated can code for the reverse complement of a cell cycle regulated miRNA. Thus, interaction between a miRNA and a (fusion protein) transcript comprising the reverse complement of the miRNA would activate the RNA-induced silencing complex (RISC), leading to degradation of the (fusion protein) transcript. Non-limiting examples of miRNAs expressed during G1 phase include miR-17/20, miR-19a, miR-24, miR-26a, miR-34a, miR-124, miR-129, and miR-137.

In other embodiments, the nucleic acid encoding the fusion protein can be operably linked to a promoter control sequence for in vitro synthesis of mRNA encoding the fusion protein. Generally, the promoter sequence is recognized by a phage RNA polymerase. For example, the promoter sequence can be a T7, T3, or SP6 promoter sequence or a variation of a T7, T3, or SP6 promoter sequence. In one embodiment, DNA encoding the fusion protein is operably linked to a T7 promoter for in vitro mRNA synthesis using T7 RNA polymerase.

In alternate embodiments, the nucleic acid encoding the fusion protein can be operably linked to a promoter sequence for in vitro expression of the fusion protein in bacterial or eukaryotic cells. Suitable bacterial promoters include, without limit, T7 promoters, lac operon promoters, trp promoters, variations thereof, and combinations thereof. Non-limiting examples of suitable eukaryotic promoter control sequences include constitutive promoters such as cytomegalovirus immediate early promoter (CMV), simian virus (SV40) promoter, elongation factor (EF1)-alpha promoter, truncated human elongation factor-1 promoter alpha (tEF1a), adenovirus major late promoter, Rous sarcoma virus (RSV) promoter, mouse mammary tumor virus (MMTV) promoter, phosphoglycerate kinase (PGK) promoter, ubiquitin promoters, actin promoters, tubulin promoters, immunoglobulin promoters, fragments thereof, or combinations of any of the foregoing, and regulated promoter control sequences such as those regulated by heat shock, metals, steroids, antibiotics, or alcohol.

In additional aspects, the nucleic acid encoding the fusion protein also can be linked to a polyadenylation signal (e.g., SV40 polyA signal, bovine growth hormone (BGH) polyA signal, etc.) and/or at least one transcriptional termination sequence (e.g., woodchuck hepatitis virus posttranscriptional regulatory element).

In various embodiments, the nucleic acid encoding the fusion protein can be present in a vector. Suitable vectors include plasmid vectors, phagemids, cosmids, artificial/mini-chromosomes, transposons, and viral vectors. In one embodiment, the DNA encoding the fusion protein is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, post-transcriptional regulatory elements, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

In embodiments in which the programmable DNA modification protein of the fusion protein is a CRISPR/Cas nuclease or a CRISPR/Cas nickase, the vector comprising the nucleic acid encoding the fusion protein can also comprise nucleic acid encoding one or more guide RNAs.

The nucleic acid encoding the fusion protein can be codon optimized for efficient translation into protein in the eukaryotic cell of interest. For example, codons can be optimized for expression in humans, mice, rats, hamsters, cows, pigs, cats, dogs, fish, amphibians, plants, yeast, insects, and so forth (see Codon Usage Database at www.kazusa.or.jp/codon/). Programs for codon optimization are available as freeware. Commercial codon optimization programs are also available.

### (III) Cells Comprising Nucleic Acids Encoding the Fusion Proteins

Still another aspect of the present disclosure encompasses a cell comprising a nucleic acid encoding any of the fusion proteins detailed above in section (I). Suitable nucleic acids are described above in section (II).

The nucleic acid encoding the fusion can be extrachromosomal in the cell. Alternatively, the nucleic acid encoding the fusion can be integrated into a chromosome (i.e., integrated into genomic DNA). The integration can be random or targeted. For example, the nucleic acid can be integrated using a lentiviral system, a retroviral system, or a targeted endonuclease system (e.g., ZFN system, CRISPR/Cas 9 system). Means for introducing nucleic acids into cells are well known in the art, and some are described below in section (IV)(a).

In one embodiment, the cell comprises nucleic acid encoding the fusion protein that is operably linked to constitutive eukaryotic promoter (e.g., tEF1a). In another embodiment, the cell comprises nucleic acid encoding the fusion protein that is operably linked to a cell cycle regulated promoter. In specific embodiments, the cell cycle regulated promoter can be a G2 promoter, an S promoter, or a G1/S promoter. The cell cycle regulated promoter can be exogenous to the cells (i.e., is introduced along with the fusion protein coding sequence). Alternatively, the cell cycle regulated promoter can be endogenous to the cells (i.e., the sequence encoding the fusion protein is targeted to integrate near an endogenous cell cycle regulated promoter sequence). In still other iterations, the cell comprises nucleic acid encoding the fusion protein that is operably linked to sequence regulated in a cell cycle dependent manner by miRNAs.

Typically, the cell cycle regulated protein of the fusion protein is selected such that the fusion protein is degraded during M phase and/or the M to G1 transition of the cell cycle. In some embodiments, the cell expresses the fusion protein during late G1 phase, S phase, and/or G2 phase of the cell cycle. For example, the operably linked cell cycle regulated sequence can be chosen to optimize expression of the fusion protein during S and/or G2 phase of the cell cycle.

The type of cell can and will vary. In various embodiments, the cell can be a human cell, a non-human mammalian cell, a stem cell, a non-human one cell embryo, a non-mammalian vertebrate cell, an invertebrate cell, a plant cell, or a single cell eukaryotic organism. The cell can be a primary cell or a cell line cells.

In some embodiments, the cell can be a human cell. Non-limiting examples of suitable human cell line cells include human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells.

In other embodiments, the cell can be a non-human mammalian cell. Non-limiting examples of suitable non-human mammalian cells include Chinese hamster ovary (CHO) cells, baby hamster kidney (BHK) cells; mouse myeloma NSO cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Nepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; and African green monkey kidney (VERO-76) cells. An extensive list of mammalian cell lines may be found in the American Type Culture Collection catalog (ATCC, Manassas, Va.).

In still other embodiments, the cell can be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells. The stem cell can be or mammalian origin.

In alternate embodiments, the cell can be non-human one cell embryo. Suitable mammalian embryos, including one cell embryos, include without limit mouse, rat, hamster, rodent, rabbit, feline, canine, ovine, porcine, bovine, equine, and primate embryos. Suitable non-mammalian embryos include amphibians, fish, fowl, and invertebrates.

In further embodiments, the cell can be a plant cell. The plant cells can be from a plant used in research (e.g., Arabidopsis, maize, tobacco) or a food plant (e.g., corn, wheat, rice, potato, cassava, soybean, yam, sorghum, etc.).

### (IV) Method for Modifying a Chromosomal Sequence or Regulating Expression of a Chromosomal Sequence

Another aspect of the present disclosure encompasses methods for using the fusion proteins disclosed herein to modify (i.e., edit) chromosomal sequences and/or regulate expression of chromosomal sequences during particular phases of the cell cycle. In embodiments in which the programmable DNA modification protein of the fusion protein has nuclease activity (i.e., is a targeting endonuclease), the chromosomal sequence cab be modified by an insertion or at least one nucleotide, a deletion of at least one nucleotide, a substitution or at least one nucleotide, and/or combinations thereof. Accordingly, the targeted chromosomal sequence can be knocked-out, can acquire a knocked-in sequence, or can be undergo a gene correction or gene conversion. In embodiments in which the programmable DNA modification protein of the fusion protein has non-nuclease activity, the targeted chromosomal sequence can undergo changes in the transcription of the targeted sequence and/or the changes in the structure of the DNA and/or associated proteins.

The method comprises introducing into the cell at least one fusion protein, as described in section (I) or nucleic acid encoding the at least one fusion protein, as described in section (II). Suitable types of cells into which the fusion protein(s) or nucleic acid encoding the fusion protein(s) can be introduced are detailed above in section (III).

In embodiments in which the programmable DNA modification protein of the fusion protein is a CRISPR/Cas nuclease or a CRISPR/Cas nickase, the method can further comprises introducing into the cell one or more guide RNAs or nucleic acids encoding one or more guide RNAs. Similarly, in embodiments in which the programmable DNA modification protein of the fusion protein is a DNA-guided Argonaute endonuclease, the method can further comprises introducing into the cell a single-stranded guide DNA.

Additionally, in embodiments in which the programmable DNA modification protein of the fusion protein has nuclease activity (i.e., is a targeting endonuclease), the method can further comprise introducing into the cell a donor polynucleotide (as detailed below) comprising at least one sequence having substantial sequence identity with a target site in the chromosomal sequence.

(a) Introducing into the Cell

The fusion protein or nucleic acid encoding the fusion protein, the optional guide nucleic acid, and the optional donor polynucleotide can be introduced into the cell by a variety of means. In some embodiments, the cell can be transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001). In other embodiments, the molecules are introduced into the cell or embryo by microinjection. For example, the molecules can be injected into the pronuclei of one cell embryos.

### (b) Culturing the Cell

The method further comprises maintaining the cell under appropriate conditions such that the fusion protein is expressed during a portion of the cell cycle. When the fusion protein is present in the cell, the DNA binding domain of the programmable DNA modification protein directs the fusion protein to a targeted site in the chromosomal sequence, wherein the programmable DNA modification protein can modify the chromosomal sequence and/or regulate expression of the chromosomal sequence.

In embodiments in which the programmable DNA modification protein of the fusion protein is a targeting endonuclease, the targeting endonuclease can introduce a double stranded break at a targeted site in the chromosomal sequence. The double stranded break can be repaired by a homology-directed repair (HDR) process or by a non-homologous end-joining (NHEJ) repair process. Because NHEJ is error-prone, nucleotide insertions and/or nucleotide deletions (i.e., indels) can occur during the repair of the break. Thus, in embodiments in which a donor polynucleotide is also introduced into the cell for targeted integration into the chromosomal sequence, repair of the break by NHEJ can hamper the targeted integration. However, since the ratio of HDR to NHEJ may be higher during G2, restricting the activity of the fusion protein to this phase of the cell cycle may increase the efficiency of genome editing by HDR and/or reduce off-target NHEJ-mediated effects. For example, in embodiments in which the fusion protein is present during the S and G2 phases, and is degraded during M and/or the M/G1 transition, repair of the double stranded break by NHEJ can be minimized. In such situations, the ratio of HDR/NHEJ is increased relative to a corresponding targeting endonuclease that is not fused to a cell cycle regulated protein. The ration or HDR/NHEJ can be increased at least 1.2-fold, at least 1.5-fold, at least 1.7-fold, or more than 1.7-fold.

In general, the cell is maintained under conditions appropriate for cell growth and/or maintenance. Suitable cell culture conditions are well known in the art and are described, for example, in Santiago et al. (2008) PNAS 105:5809-5814; Moehle et al. (2007) PNAS 104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

### (c) Optional Donor Polynucleotide

The donor polynucleotide comprises at least one sequence having substantial sequence identity with a target site in the chromosomal sequence. The donor polynucleotide also generally comprises a donor sequence. The donor sequence can be an exogenous sequence. As used herein, an “exogenous” sequence refers to a sequence that is not native to the cell, or a chromosomal sequence whose native location in the genome of the cell is in a different chromosomal location. For example, the donor sequence can comprise an exogenous protein coding gene, which can be operably linked to a promoter control sequence such that, upon integration into the cell, the cell expresses the protein coded by the integrated gene. Alternatively, the exogenous protein coding sequence can be integrated into the chromosomal sequence such that its expression is regulated by an endogenous promoter control sequence. Integration of an exogenous gene into the chromosomal sequence is termed a “knock in.” In other embodiments, the exogenous sequence can be a transcriptional control sequence, another expression control sequence, an RNA coding sequence, and so forth.

In some embodiments, the donor sequence of the donor polynucleotide can be a sequence that is essentially identical to a portion of the chromosomal sequence at or near the targeted site, but which comprises at least one nucleotide change. Thus, the donor sequence can comprise a modified version of the wild type sequence at the targeted site such that, upon integration or exchange with the chromosomal sequence, the sequence at the targeted chromosomal location comprises at least one nucleotide change. For example, the change can be an insertion of one or more nucleotides, a deletion of one or more nucleotides, a substitution of one or more nucleotides, or combinations thereof. As a consequence of the integration of the modified sequence, the cell can produce a modified gene product from the targeted chromosomal sequence.

As can be appreciated by those skilled in the art, the length of the donor sequence can and will vary. For example, the donor sequence can vary in length from several nucleotides to hundreds of nucleotides to hundreds of thousands of nucleotides.

In some embodiments, the donor sequence in the donor polynucleotide is flanked by an upstream sequence and a downstream sequence, which have substantial sequence identity to sequences located upstream and downstream, respectively, of the targeted site in the chromosomal sequence. Because of these sequence similarities, the upstream and downstream sequences of the donor polynucleotide permit homologous recombination between the donor polynucleotide and the targeted chromosomal sequence such that the donor sequence can be integrated into (or exchanged with) the chromosomal sequence.

The upstream sequence, as used herein, refers to a nucleic acid sequence that shares substantial sequence identity with a chromosomal sequence upstream of the targeted site. Similarly, the downstream sequence refers to a nucleic acid sequence that shares substantial sequence identity with a chromosomal sequence downstream of the targeted site. As used herein, the phrase “substantial sequence identity” refers to sequences having at least about 75% sequence identity. Thus, the upstream and downstream sequences in the donor polynucleotide can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with sequence upstream or downstream to the targeted site. In an exemplary embodiment, the upstream and downstream sequences in the donor polynucleotide can have about 95% or 100% sequence identity with chromosomal sequences upstream or downstream to the targeted site. In one embodiment, the upstream sequence shares substantial sequence identity with a chromosomal sequence located immediately upstream of the targeted site (i.e., adjacent to the targeted site). In other embodiments, the upstream sequence shares substantial sequence identity with a chromosomal sequence that is located within about one hundred (100) nucleotides upstream from the targeted site. Thus, for example, the upstream sequence can share substantial sequence identity with a chromosomal sequence that is located about 1 to about 20, about 21 to about 40, about 41 to about 60, about 61 to about 80, or about 81 to about 100 nucleotides upstream from the targeted site. In one embodiment, the downstream sequence shares substantial sequence identity with a chromosomal sequence located immediately downstream of the targeted site (i.e., adjacent to the targeted site). In other embodiments, the downstream sequence shares substantial sequence identity with a chromosomal sequence that is located within about one hundred (100) nucleotides downstream from the targeted site. Thus, for example, the downstream sequence can share substantial sequence identity with a chromosomal sequence that is located about 1 to about 20, about 21 to about 40, about 41 to about 60, about 61 to about 80, or about 81 to about 100 nucleotides downstream from the targeted site.

Each upstream or downstream sequence can range in length from about 20 nucleotides to about 5000 nucleotides. In some embodiments, upstream and downstream sequences can comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2800, 3000, 3200, 3400, 3600, 3800, 4000, 4200, 4400, 4600, 4800, or 5000 nucleotides. In exemplary embodiments, upstream and downstream sequences can range in length from about 500 to about 1500 nucleotides.

Donor polynucleotides comprising the upstream and downstream sequences with sequence similarity to the targeted chromosomal sequence can be linear or circular. In embodiments in which the donor polynucleotide is circular, it can be part of a vector (detailed above). For example, the vector can be a plasmid vector.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “endogenous sequence” refers to a chromosomal sequence that is native to the cell.

The term “exogenous,” as used herein, refers to a sequence that is not native to the cell, or a chromosomal sequence whose native location in the genome of the cell is in a different chromosomal location.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

As various changes could be made in the above-described cells and methods without departing from the scope of the invention, it is intended that all matter contained in the above description and in the examples given below, shall be interpreted as illustrative and not in a limiting sense.

### EXAMPLES

The following examples detail certain embodiments of the disclosure.

### Example 1

### Preparation of Cas9 Linked to Geminin

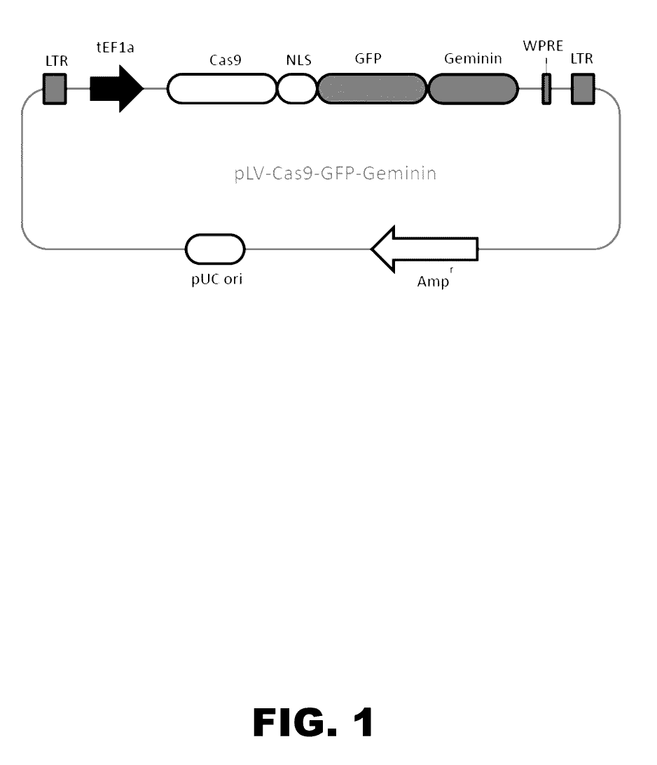

To limit expression of Cas9 to S/G2 phases of the cell cycle, Cas9 was fused to geminin, a protein that is degraded during M phase. For this, Cas9 from Streptococcus pyogenes was fused to green fluorescent protein (GFP) and geminin with Cas9 at the N-terminus (FIG. 1). The fusion also comprised a nuclear localization signal (NLS) and linkers (e.g., 2×GS linkers) flanking the GFP domain (e.g., Cas9-NLS-Linker-GFP-Linker-Geminin). The DNA sequence of the fusion is presented in Table 1 and the protein sequence is presented in Table 2.

TABLE 1
DNA sequence of Cas9-NLS-GFP-Geminin Fusion

| ID | DNA sequence (5′ - 3′) |
|---|---|
| Cas9 | atggacaagaagtacagcatcggcctggacatcggcaccaactctgtgggctgggccgtgatcaccgacgactac |
| | aaggtgcccagcaagaaattcaaggtgctgggcaacaccgaccggcacagcatcaagaagaacctgatcggc |
| | gccctgctgttcggctctggcgaaacagccgaggccacccggctgaagagaaccgccagaagaagatacacca |
| | gacggaagaaccggatctgctatctgcaagagatcttcagcaacgagatggccaaggtggacgacagcttcttcc |
| | acagactggaagagtccttcctggtggaagaggataagaagcacgagcggcaccccatcttcggcaacatcgtg |
| | gacgaggtggcctaccacgagaagtaccccaccatctaccacctgagaaagaagctggccgacagcaccgac |
| | aaggccgacctgagactgatctacctggccctggcccacatgatcaagttccggggccacttcctgatcgagggcg |
| | acctgaaccccgacaacagcgacgtggacaagctgttcatccagctggtgcagatctacaatcagctgttcgagga |
| | aaaccccatcaacgccagcagagtggacgccaaggccatcctgagcgccagactgagcaagagcagacggct |
| | ggaaaatctgatcgcccagctgcccggcgagaagcggaatggcctgttcggcaacctgattgccctgagcctggg |
| | cctgacccccaacttcaagagcaacttcgacctggccgaggatgccaaactgcagctgagcaaggacacctacg |
| | acgacgacctggacaacctgctggcccagatcggcgaccagtacgccgacctgtttctggccgccaagaacctgt |
| | ccgacgccatcctgctgagcgacatcctgagagtgaacagcgagatcaccaaggcccccctgtccgcctctatgat |
| | caagagatacgacgagcaccaccaggacctgaccctgctgaaagctctcgtgcggcagcagctgcctgagaagt |
| | acaaagagattttcttcgaccagagcaagaacggctacgccggctacatcgatggcggagccagccaggaaga |
| | gttctacaagttcatcaagcccatcctggaaaagatggacggcaccgaggaactgctcgtgaagctgaacagaga |
| | ggacctgctgcggaagcagcggaccttcgacaacggcagcatcccccaccagatccacctgggagagctgcac |
| | gccattctgcggcggcaggaagatttttacccattcctgaaggacaaccgggaaaagatcgagaagatcctgacct |
| | tcagaatcccctactacgtgggccctctggccaggggaaacagcagattcgcctggatgaccagaaagagcgag |
| | gaaaccatcaccccctggaacttcgaggaagtggtggacaagggcgccagcgcccagagcttcatcgagcggat |
| | gaccaacttcgataagaacctgcccaacgagaaggtgctgcccaagcacagcctgctgtacgagtacttcaccgt |
| | gtacaacgagctgaccaaagtgaaatacgtgaccgagggaatgcggaagcccgcctttctgagcggcgagcag |
| | aaaaaggccatcgtggacctgctgttcaagaccaaccggaaagtgaccgtgaagcagctgaaagaggactactt |
| | caagaaaatcgagtgcttcgacagcgtggaaatcagcggcgtggaagatcggttcaacgcctccctgggcgccta |
| | tcacgatctgctgaaaattatcaaggacaaggacttcctggacaatgaggaaaacgaggacattctggaagatatc |
| | gtgctgaccctgacactgtttgaggaccggggcatgatcgaggaacggctgaaaacctatgcccacctgttcgacg |
| | acaaagtgatgaagcagctgaagcggcggagatacaccggctggggcaggctgagccggaagctgatcaacg |
| | gcatccgggacaagcagtccggcaagacaatcctggatttcctgaagtccgacggcttcgccaacagaaacttcat |
| | gcagctgatccacgacgacagcctgacctttaaagaggacatccagaaagcccaggtgtccggccagggacact |
| | ctctgcacgagcagatcgccaatctggccggatcccccgccattaagaagggcatcctgcagacagtgaagattgt |
| | ggacgagctcgtgaaagtgatgggccacaagcccgagaacatcgtgatcgaaatggccagagagaaccagac |
| | cacccagaagggacagaagaacagccgcgagagaatgaagcggatcgaagagggcatcaaagagctgggc |
| | agccagatcctgaaagaacaccccgtggaaaacacccagctgcagaacgagaagctgtacctgtactacctgca |
| | gaatgggcgggatatgtacgtggaccaggaactggacatcaaccggctgtccgactacgatgtggaccacattgtg |
| | ccccagtccttcatcaaggacgactccatcgataacaaagtgctgactcggagcgacaagaaccggggcaagag |
| | cgacaacgtgccctccgaagaggtcgtgaagaagatgaagaactactggcgccagctgctgaatgccaagctga |
| | ttacccagaggaagttcgacaatctgaccaaggccgagagaggcggcctgagcgaactggataaggccggcttc |
| | attaagcggcagctggtggaaacccggcagatcacaaagcacgtggcacagatcctggactcccggatgaaca |
| | ctaagtacgacgagaacgacaaactgatccgggaagtgaaagtgatcaccctgaagtccaagctggtgtccgact |
| | tcagaaaggatttccagttttacaaagtgcgcgagatcaacaactaccaccacgcccacgacgcctacctgaacg |
| | ccgtcgtgggaaccgccctgatcaaaaagtaccctaagctggaaagcgagttcgtgtacggcgattacaaggtgta |
| | cgacgtgcggaagatgatcgccaagagcgagcaggaaatcggcaaggctaccgccaagtacttcttctacagca |
| | acatcatgaactttttcaagaccgagatcacactggccaacggcgagatcagaaagcggcctctgatcgagacaa |
| | acggcgaaaccggggagatcgtgtgggataagggccgggattttgccacagtgcggaaagtgctgtccatgcccc |
| | aagtgaatatcgtgaaaaagaccgaggtgcagaccggcggcttcagcaaagagtctatcctgcccaagaggaac |
| | tccgacaagctgatcgccagaaagaaggattgggaccctaagaagtacggcggctttgacagccccaccgtggc |
| | ctactctgtgctggtggtggccaaagtggaaaagggcaagtccaagaaactgaagagtgtgaaagagctgctggg |
| | gatcaccatcatggaaagaagcagcttcgagaagaatcccatcgactttctggaagccaagggctacaaagaagt |
| | gaaaaaggacctgatcatcaagctgcctaagtactccctgttcgagctggaaaacggccggaagcggatgctggc |
| | ttctgccggcgaactgcagaagggaaacgagctggccctgccctccaaatatgtgaacttcctgtacctggccagc |
| | cactatgagaagctgaagggctcccccgaggataatgagcagaaacagctgtttgtggaacagcacaagcacta |
| | cctggacgagatcatcgagcagattagcgagttctccaagcgcgtgatcctggccgatgccaacctggacaaggt |
| | gctgagcgcctacaacaagcaccgggataagcccatcagagagcaggccgagaatatcatccacctgtttaccct |
| | gaccaacctgggagcccctgccgccttcaagtactttgacaccaccatcgaccggaagaggtacaccagcacca |
| | aagaggtgctggacgccaccctgatccaccagagcatcaccggcctgtacgagacacggatcgacctgtctcag |
| | ctgggaggcgac (SEQ ID NO: 9) |
| NLS | cccaagaaaaagcgcaaagtg (SEQ ID NO: 10) |
| Linker | ggcggctccggcggcggcagcggc (SEQ ID NO: 11) |
| GFP | agcgggggcgaggagctgttcgccggcatcgtgcccgtgctgatcgagctggacggcgacgtgcacggccacaa |
| | gttcagcgtgcgcggcgagggcgagggcgacgccgactacggcaagctggagatcaagttcatctgcaccaccg |
| | gcaagctgcccgtgccctggcccaccctggtgaccaccctctgctacggcatccagtgcttcgcccgctaccccga |
| | gcacatgaagatgaacgacttcttcaagagcgccatgcccgagggctacatccaggagcgcaccatccagttcca |
| | ggacgacggcaagtacaagacccgcggcgaggtgaagttcgagggcgacaccctggtgaaccgcatcgagct |
| | gaagggcaaggacttcaaggaggacggcaacatcctgggccacaagctggagtacagcttcaacagccacaa |
| | cgtgtacatccgccccgacaaggccaacaacggcctggaggctaacttcaagacccgccacaacatcgagggc |
| | ggcggcgtgcagctggccgaccactaccagaccaacgtgcccctgggcgacggccccgtgctgatccccatcaa |
| | ccactacctgagcactcagaccaagatcagcaaggaccgcaacgaggcccgcgaccacatggtgctcctggag |
| | tccttcagcgcctgctgccacacccacggcatggacgagctgtacagggc (SEQ ID NO: 12) |
| Linker | ggcggctccggcggcggcagcggc (SEQ ID NO: 11) |
| Geminin | atgaatcccagtatgaagcagaaacaagaagaaatcaaagagaatataaagaatagttctgtcccaagaagaa |
| 1-110 | ctctgaagatgattcagccttctgcatctggatctcttgttggaagagaaaatgagctgtccgcaggcttgtccaaaag |
| | gaaacatcggaatgaccacttaacatctacaacttccagccctggggttattgtcccagaatctagtgaaaataaaa |
| | atcttggaggagtcacccaggagtcatttgatcttatgattaaagaaaatccatcctctcagtattggaaggaagtggc |
| | agaaaaacggagaaaggcgctg (SEQ ID NO: 13) |
| Stop | tgatga |
| codons | |

TABLE 2
Protein Sequence of Cas9-NLS-GFP-Geminin Fusion*

| | |
|---|---|
| MDKKYSIGLDIGTNSVGWAVITDDYKVPSKKFKVLGNTDRHSIKKNLIGA | 50 |
| LLFGSGETAEATRLKRTARRRYTRRKNRICYLQEIFSNEMAKVDDSFFHR | 100 |
| LEESFLVEEDKKHERHPIFGNIVDEVAYHEKYPTIYHLRKKLADSTDKAD | 150 |
| LRLIYLALAHMIKFRGHFLIEGDLNPDNSDVDKLFIQLVQIYNQLFEENP | 200 |
| INASRVDAKAILSARLSKSRRLENLIAQLPGEKRNGLFGNLIALSLGLTP | 250 |
| NFKSNFDLAEDAKLQLSKDTYDDDLDNLLAQIGDQYADLFLAAKNLSDAI | 300 |
| LLSDILRVNSEITKAPLSASMIKRYDEHHQDLTLLKALVRQQLPEKYKEI | 350 |
| FFDQSKNGYAGYIDGGASQEEFYKFIKPILEKMDGTEELLVKLNREDLLR | 400 |
| KQRTFDNGSIPHQIHLGELHAILRRQEDFYPFLKDNREKIEKILTFRIPY | 450 |
| YVGPLARGNSRFAWMTRKSEETITPWNFEEVVDKGASAQSFIERMTNFDK | 500 |
| NLPNEKVLPKHSLLYEYFTVYNELTKVKYVTEGMRKPAFLSGEQKKAIVD | 550 |
| LLFKTNRKVTVKQLKEDYFKKIECFDSVEISGVEDRFNASLGAYHDLLKI | 600 |
| IKDKDFLDNEENEDILEDIVLTLTLFEDRGMIEERLKTYAHLFDDKVMKQ | 650 |
| LKRRRYTGWGRLSRKLINGIRDKQSGKTILDFLKSDGFANRNFMQLIHDD | 700 |
| SLTFKEDIQKAQVSGQGHSLHEQIANLAGSPAIKKGILQTVKIVDELVKV | 750 |
| MGHKPENIVIEMARENQTTQKGQKNSRERMKRIEEGIKELGSQILKEHPV | 800 |
| ENTQLQNEKLYLYYLQNGRDMYVDQELDINRLSDYDVDHIVPQSFIKDDS | 850 |
| IDNKVLTRSDKNRGKSDNVPSEEVVKKMKNYWRQLLNAKLITQRKFDNLT | 900 |
| KAERGGLSELDKAGFIKRQLVETRQITKHVAQILDSRMNTKYDENDKLIR | 950 |
| EVKVITLKSKLVSDFRKDFQFYKVREINNYHHAHDAYLNAVVGTALIKKY | 1000 |
| PKLESEFVYGDYKVYDVRKMIAKSEQEIGKATAKYFFYSNIMNFFKTEIT | 1050 |
| LANGEIRKRPLIETNGETGEIVWDKGRDFATVRKVLSMPQVNIVKKTEVQ | 1100 |
| TGGFSKESILPKRNSDKLIARKKDWDPKKYGGFDSPTVAYSVLVVAKVEK | 1150 |
| GKSKKLKSVKELLGITIMERSSFEKNPIDFLEAKGYKEVKKDLIIKLPKY | 1200 |
| SLFELENGRKRMLASAGELQKGNELALPSKYVNFLYLASHYEKLKGSPED | 1250 |
| NEQKQLFVEQHKHYLDEIIEQISEFSKRVILADANLDKVLSAYNKHRDKP | 1300 |
| IREQAENIIHLFTLTNLGAPAAFKYFDTTIDRKRYTSTKEVLDATLIHQS | 1350 |
| ITGLYETRIDLSQLGGDPKKKRKVGGSGGGSGSGGEELFAGIVPVLIELD | 1400 |
| GDVHGHKFSVRGEGEGDADYGKLEIKFICTTGKLPVPWPTLVTTLCYGIQ | 1450 |
| CFARYPEHMEMNDFFKSAMPEGYIQERTIQFQDDGKYKTRGEVKFEGDTL | 1500 |
| VNRIELKGKDFKEDGNILGHKLEYSFNSHNVYIRPDKANNGLEANFKTRH | 1550 |
| NIEGGGVQLADHYQTNVPLGDGPVLIPINHYLSTQTKISKDRNEARDHMV | 1600 |
| LLESFSACCHTHGMDELYRAGGSGGGSGMNPSMKQKQEEIKENIKNSSVP | 1650 |
| RRTLKMIQPSASGSLVGRENELSAGLSKRKHRNDHLTSTTSSPGVIVPES | 1700 |

SENKNLGGVTQESFDLMIKENPSSQYWKEVAEKRRKAL** (SEQ ID NO: 14)
*NLS in bold, GS linkers underlined.

### Example 2

### Analysis of Cas9-GFP-Geminin Fusion

The sequence encoding the Cas9-Geminin fusion protein was operably linked to a tEF1alpha promoter sequence for expression in eukaryotic cells (see FIG. 1). The use of lentiviral formats allows for the creation of stable cell lines or pooled populations of cells expressing Cas9-Gem fusions. Initial experiments will compare nuclease activities of Cas9-Gem and Cas9 at known guide RNA (gRNA) target sites to determine if geminin fusion has any impact on nuclease activity. Example target sites for testing include KRAS (5′-TAGTTGGAGCTGGTGGCGTAGG-3′; SEQ ID NO: 15), HPRT1 (5′-TTATATCCAACACTTCGTGGGG-3′; SEQ ID NO: 16), and others (PAM underlined). Transfected cell populations will be treated with gRNA and analyzed by microscopy and FACS to observe GFP expression and to assess if GFP signal corresponds to G2/S cell cycle timing as previously observed for GFP-geminin fusions (Sakaue-Sawano et al., 2008). Using nuclease sensitive reporter plasmids, experiments will also be attempted to observe Cas9 cutting activity and assess if cutting activity and Cas9-GFP-geminin expression are synchronized in the G2 phase of the cell cycle.

As an alternate or combined approach, Cas9 or Cas9-Geminin can be placed under control of promoters associated with transcripts present in phase G2 of the cell cycle. Exact timing of promoter activity may be critical to achieving beneficial effects such as increased HR/NHEJ ratios and reduced off-target effects, thus several different promoter regions will be chosen from the published literature. (Whitfield et al., 2002). An example promoter sequence is listed below in Table 3 for human gene TOP2A (hg38_chr17:40380861-40390549).

TABLE 3
DNA sequence of promoter region of human TOP2A gene.

>hg38_chr17: 40380861-40390549_TOP2A-promoter-region
gcagtctattcaccctcctcagtgtcatacctttctgctgtcttctgattgagttctctgcctacactctcctccaggtgatagttgtagcctttac
agcaaaccagtggacaagaagcatcagggtctttggaaattttgctgtgcattggaccagtaaaagtaattccagatctgaagacagc
ttgactttggcttatttttactgattcctatttgtgtttttcagaaagagctacttgatcaccagctctagaagtatcaggagttacaattatccaa
tcttatgcaaattggctggtgggctgcaaagcttgtgtactttttgcagtgggggttgtacaaacagaaaaataaagaatacaagggtcg
ggccaggcacggtctctcatgcctgtaatcccagcactttgggaggtcgaggtgagaggatcacttgaaaccaggagttcgagacca
gcatggccagcttggtgaaaccccgtctgtactaaaaatacaaaaattagctgggcatggtggcacacgcctgtagtcccagctactc
gggaggctgagacaggagaattgcttgaacctgggaggtggaggttgcagtgagctgagattgtgccactgcactccagcctgggc
gacagagtgagactgtctcaaaacaaaaaacaaggctcttctgaagacgctttaatgaaaatcattatttcttagtcaccccaagagc
atgaatttgatgtggttgggaactcaagctaaatattgtgaaggtgtaactctgtgttgacctctagccatgcagctcagttgttttgcaaact
gtcctgatttcccacagatgacttgtcctactgaggacacctatcagtaggtcagagagcagctttgtgagccttcctgctggtacccaga
agtgagtttgtgcccactaattttttagcattttaattcctcgcaacagaagagactggcaaaactcaacaattctctgtatttatttatgtatttt
tgagacaaggtcttgccctatcacccaggctgatgtgcagtggcacgatcatggctcattgcagctttgacctcatgggtttaagggattc
tcccacctcagcctcctgagtagctgggaccacaggtgcaagccaccatgccctattaacttttttttttttttaagacagggttttgctgtctg
tcacccaggctggagtacagtggtgcgatcttggctcactgcaacctccacctcctgggttcaaatgattctcctgtctcagctgaccga
gtagctggtattacaggcatgtgccaccacacccagctaatttttgtatttttagtggagatggggtttaaccatgttggccaggctggtctc
gaactcttgacctcaagtgttccacctgtcttggcctcccaaaatgttgggattacaggtgtgaactactgcacccagacaagaaaaca
catacttatttttataaactataggaaagcacaaagaaaacaaaaatcatcgaaatctcattctccagataaaagcagctgacattttgc
tgcgacttgcaaaatgcctttggattcagataacagtggttctgaaactttagcgtgcatcagaattaactggagggcttgttaaaacagt
gcttctgagtcagaagttttggagtggagccgataatttgaatttctttctttctttcttttttttttttttttgagacagtttccctcttgtttcccaggct
ggagtgcattggcacaatcttggctcactgcaacctccacctcctaggttcaagcaattcttctgcctcagcctctcgagtagctgggatt
acagatgcccgccaccatgcccagctaattttttgtatttctagtagagacagggtttcactgttggctacgctggtcttgaactcctgacct
caggcaatccacccatgtcagcctcctaaggtgctgggattacaggcatgagccaccacatccagctgataatttgaatttctaagaa
gctcccaggtgtccctgacactgttggtccaggtatcatacattgagaagcactggatatgtgcaccttggctgttccaagtagggtctgc
aaccagaggcattgacatcattttgggaacttgtaatgcagaatctcaggccccagctcagacctactgaatcataatctgtaatttaata
agatccctaaaaaatttttaagcaccaggcacggtggctcacgcgtgtaatcccagcactttgggaggccaagcgggtggatcacga
ggtcaggagttcaagaccagcctggccaagatggtgaaaccctgtctctactaaaaatacaaaaattagccgggtgtggcggtggg
cacctgtaatcccagctactcgggaggctgaggcagagaattgcttgaacctgggaggcagaggttgcagttagccgagatcgtgcc
actgtattccaacctaggtgacagagtgagactccatctcaaaaaaaaaaaaaaaaaaaatttttttaagcacaggtttgagaaggat
tggtttatattttaagcctcatagtatataacagttactccccccaccatattgaggtagaatttacacatagtgcaccattttataatgtataa
tttgatgagttttgacaaaatgatactaaatagttttgtacccttttgtctctctacccaacataatgaggactttcctgtagtattagatgttttgg
aaaaacatgacttctaatggctgtacaatacattgtaggtaaggatgttccagtttaaccaattcttcttttatttatttatttatttatttttgagac
agagtctcttgctgttgcccagtctggactatagtggcgcagtcttggctcactgcaacctgcacttcctgggttcaagcgagtcttgtgtct
cagcctcccaagtagctgagactacaggtgtgcaccaccacactcaggtaatttttgtattttcagtagagacagggtttcgacatgttgc
ccaggctggtctcctgagctcaggcaatctgcctgcctaggcctcccaaagtgctgggattacaggcgtgagccactgtacctggccc
agtttaaccaattcttctattgtgagacatctatgttgttcccaatttctcaccagtgtaaataatgcttcaatgaatgcttttggacttaaatgttt
tcgtttggactttaacatatttttccacagctaaattactgaggaaagggtacgggacaggcaagaacaggtatccattactcaagaatg
aaaagttaatgaattaaatttttctgtttgggtttcaggaaaaatggctagaaatcattaaaaaaaaaatccattgcagcagaaacagtg
ggatgcactgtatcttaaaaacaaaaagggccaggctgggcacagtggctcacgcctgtaatcccagcactttgggaggctgagatg
ggtggatcacctgaggtcaggaactcaagaccagcccggccaaactggtaaaactctgcctttactaaaaatacaaaaattagctgg
gtgtggtggcgtgcgcttgtaatcccaggtactcgggaggctgaggcaggagaatcgcttgaacctgggaggcggaggttgcagtga
gccgaagctgtgccattccactccagcctgggcgacagaacgagactcaatcttaaaaaaaaaaaaaaaagaaaaaagccggg
agtggtggcaggtgcctgcaatcctaggtacttgggaggctgaggcaggagaattgcttgagcccaggaggcggaggttgcagtga
gctgaaatggtgccactgcactccagcctgggcagcagagcaagactctgtctcatggaaaaaataaaataaaaaaaaaaagact
cagtaaacttactgttgaatcctttaccaattaatgcaacttttgagtcttttctcaatagccattcttttgtaattcataacttatatgtatttaagg
aatgtttcatacacataggaaataaccacattctataaagggtctaaatacataaaactatcacgtttattagcaaatctttatatcctttaat
gtgtcagtagcttaagaaataatgaaggccgaaggccaggcgcagtggctcacgcctgtaatcccagcactttgggaggccgaggc
gggtggatcacgaggtcaggagatcgagaccatcatggctaacatggtgaaaccctgtctctactaaaaatataaaaaattagccag
gcgtggtggcaggcggctgtagtcccagctacttgggaggctgaggcaggagaatcgcttgaacctgggaggcggaggttgcagtg
agctgagattgtgccactgcactccagcctgggcggcagagtcagattccatttcaaaaaaaaaataaataaataaaagaaaaaaa
aaagaaataatgaataggcctggcatggtggctcacgcctgtaatcgcagctctttgggaggttgaggcaggtggatcacttgagccc
aggagttccagaacagccggggcaacatagtgagaccctgcctctacaaaaaatacaaaaattagccaggtgtggtggtgtgtacc
tgtggtcccagctatttgggaggctgaggcaggaggatcgcttgagcccaggaggcagaggttgcagtgggccgagattgagccac
tgcactccagcctggatggtagagtgaaaccttgtctcaaaaaaagaaaaaaagaaaaaaaagagtcaaggaaacattatccgctt
tcagttagcaaggtctttactcatcaggaaatgtaaaacttctactttcaaaagagaactattggccgggcgcggtggctcaggcctgta
atcccagcactttgggacgcggaggcaggcggattgcctgagctcagaccagcctgggcaacatggtgaaaccccatctctactaa
aaatacaaaaaatttaagctgggcgtggtggctcatgcctgtaatcccagcactttgggtgtctgaagtgggacgatcacttgaggtca
ggaattcgagaccagcctggacaacatggtgaaactccatctctactaaaaatacaaaaattaactgtaatttttgtattccctgtgatcc
cagccacttgggaggctgaggcatgagaatcacttgaaccaggcaggcggaggttatagtgagccgagatcgtgccactgcactcc
agcctgggtgatagagcaagacaagactttatcccccaaaaaacaaaaaaacccagaaaatcccacaaataaaaacacaaaga
attagccaggcatggcagtaggcgcctgtagtcccagctacttgggaggctgaggcatgagaattgcttgaccttgggaggcagaaa
gcagagaattgcagtgagctgagatcgtaccactgcactccagcctgggtgccaaaatgagattctatctccaaaaaaaaaaaaaa
ggaaaaatatttgattcttttactttctaaaaagagtttacatactttcctcccactatttattttgtaaacaactggcatatttaccagatgggg
atttcatctttgatttgtaatctgcttttttccacttggcaatgtcgtgaacatctatcttttcatgtcaataaatgtcaataaataaacagtataga
tgatcattcatttttttttttttttgagacagtcttgctctgttgcccaggctggagtgcagtgccatcatggctcactgcagccccctgggctca
agcaatactcctgcctcagccttccaagtagctgggaccacaggcatgcaccaccatgtccagctgatttttacctttttttttgtagagatg
ggggtctcactacgttgcccaggctggtctcaaactcctgggctcaagcaatcttcccacttcagcctcccaaagtgctgggaatacat
gtatgaaccactgtgcctggtctacctgatcattttttttttcttgatggaatttcactcatgttacccaggatggagtgcaatagcacgatcttg
gctcactgcaacctccacctcctgggttcaagcgattctcctgcctcagcctcctgagtagttgggattacaggtgcacgccaccacac
ctggctaatttttgtatttttagtagagacggggtttcaccatgttggtcaggctggtctcgaactcctgacctcgtggtctgcttgccttgggct
cccaaagtgctgggattacaggcgtgagccactgcgcctggcctacatgatcattcctaataggcacctggtattccatatttaccatttta
accttttggacatttaggttattttccattttattattacagcaacttcaataagcatctttgcatgtggctttgttttgatatagttgtacattcacat
agttttaagaaatggatcaggccgggcatggtggctcacgcctgtaatcccagcactttgggaggctgaggtgggcggatcacaagg
tcaggagtttgagaccagccgggccaacatggtgaaaccctgtctctactaaaaatacaaaaattagctgggcgtggtggcatgcac
ctataatgccagctactcgggaggctgaggcaggagaatcgtttgtacccgggaggcagaagttgcaatgagtcaagatggcccca
gtgcactccagcctgggcgacagagcaagactctgtcccagaaaaaaaaaaaaagaaatggatcagaaacaaggactctttctg
aaaggaaaaaaaaaagaatggagatccatcgtatactttgcccatttcccaattttgcaaaattatatagtaaccagaatacttacattg
aagcaacccattgatcttactcagatttacttatactcatatttgtgtgtgtttacatagttttttgcatgtctgattcttctgtcaaacgaaattcct
ttttttttttttttttgagacagggacttgctcaggctggaatgcagtggcacaatctctggtcactgtaacctctgcttcctgggctcaagcaat
cttccctccttggcctcccaaactgctgggattacaggtgtgagccaccatgcctggcccagatttctttgaaagggctaattcctccatat
ctttgtcaacactacttttgggttttgttcagtttatccctctgtaactcaagattactttttttatagttactttttaaatagtttttgacatttaaatattt
catctatttgaacttaattttggtgtaaggtgtgaaagagatttatctgattttttttctaaatggattagccagttgcctcaatatatcttactgat
accatcaagtagttgactaggttatcaaaatagttgttaaaggaaggtatcattaaaaaaaaaagatacatgcatatttactgatcaagt
gtggtggagatgaagaacttagtcctcatgtataaaatctcaataaagagtctttggccttaattaggtcttaatgcctatctcttggacttat
caccttagccagaggctgtaaggtctgtcacaatatgattggaatgcttctgaaagggaagtgaagactatattttagaataaggaaaa
gggtgtagtgtgtgttttaaaagaggcattctatgggttgcaatgtttagaacattttattaaagtacaaaattgttggaatttagctaataga
aaaacatagtaaatatttacaaaaacgttgataacattactcaagtcacacacatataacaatgtagacaggtcttaacaaagtttaca
aattgaaattatggagatttcccaaaatgaatctaatagctcattgctgagcatggttatcaatataacatttaagatcttggatcaaatgtt
gtccccgagtcttctgcaatccagtcctcttagaaattggtttctctctttgggagattcagactcagaggcagccagaggggacaggtc
aagagctgaaataatcacataactactctaattttcttcattctattgactgtgtcaagttatagacacagccaaagtgtttttcttcggcctct
gatgatttgagaagatgaagaacatgagcaatttctcattgcttaaagaaaaacttggcacataagaggctgagtgtagtagagtatct
gtactagaaccataaagttctatctgatggtaaattatgtataaaactaagataaaacagataattatgctctatctcatatctactgaaag
tagaaaaggaggaagagtgacacttttaaatcaaactgctctagttttagcttagtggatggttaataaacacactgctttacgctgaagt
gatcagatagctatttctacagttcagaagaacttaaaaatcaggttttaaagacaaaagaaagcagactcaaaacacagacaaag
cagagaagaaaacaatgcccatgagatggtcactatttagacagtattataaaaagctaaagaacacttgggctttacttcactttgatg
tcttgtactaaaaacaccttccccaaactaaattcagaggggaggaagttaagagcttcaggtaactttaaaaccagtcttgggcttggt
aagataattacttaaaataatcgcctcacattttaaaacagatcatcttcatctgactcttccaggtactttataggtttctttgcccgtacaga
ttttgcccgaggagccacagctgagtcaaagtccatatggaagtcatcactctcccccttggatttctaaaagagaaaagcccaggta
acttgcacattgtaaatctgacaacataattgtaatgtaaaaaaatgtatcaagacactatattcaaggagttttctattttctaccaagtaa
taagaagcagatctaaggccaactcttccattgcccaaataagtggcatatttaactttgttaaaactaaatatgtacagtaaaagctaa
cagaatatgagagttaattttcttaaagatatgccaaatttttaagagcaatggcttagttacgtgtttcagaacatctacagcaaaagga
ctgactaggatcaacactcaccttgcttgtgactgctttcgaaacaattttctcaaaattagagtcagaatcatcagaagtggatggcttcc
ttttgcggcgattcttggttttggcaggatcaggcttttgagagacaccagaattcaaagctggatcccttttagttccttttggggcagccctt
tttttggcaccggtagtggaggtggaagactgacctgcaattcaatacaggcatttgtcacagctgctctttttttgagatggggtctcactc
tatcgtccaggctggagtgcagtggtgttatctcggctcactgcaacctctgcctcctgggttcaagcgattctcctgcctcagcctcctga
gtagctgggattacaggcgtgtgccaccacacccggctaattttttgtatttttagtagagatgggattccaccatgttggtcaagctggtct
caaactcctgacctcaggtgatccactcgcctcggcctcccaaagtgctgggattacaggcatgagcaaccgcgcctgacctagtca
cagccactcttagatgaattgttctcattgcgaactttcttcagcaatgtgatg (SEQ ID NO: 15).

### Example 3

### Expression of Cas9-GFP-Gemimin Fusion Protein is Cell Cycle Dependent

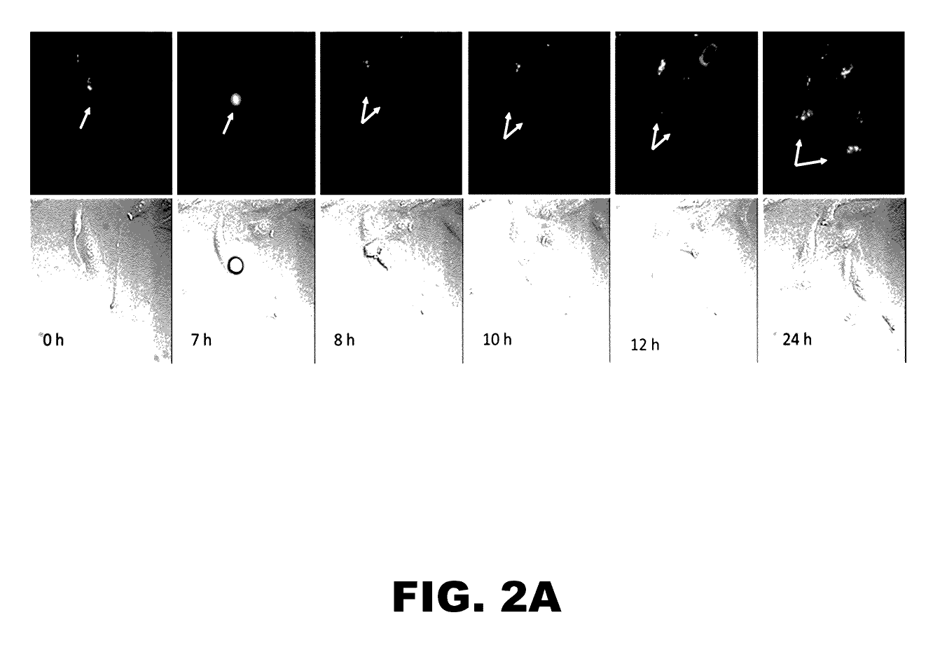

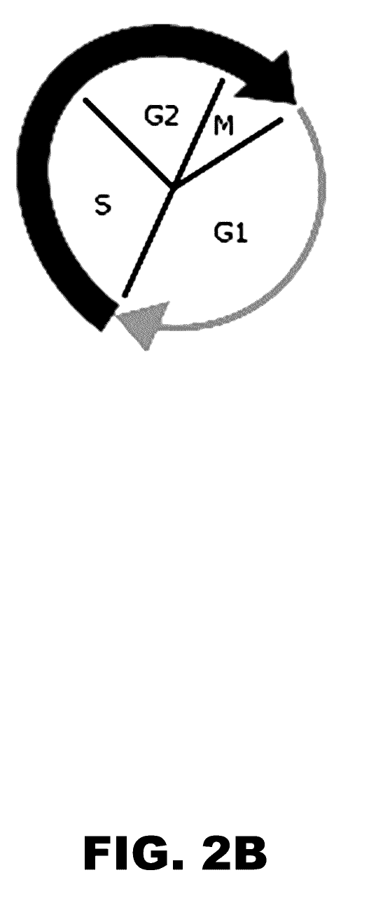

To determine whether expression of Cas9-GFP-Gemimin fusion protein is cell cycle dependent in human cells, U2OS cells were transfected by Amaxa nuclefection with 4 μg of Cas9-GFP-Gemimin plasmid DNA. Twenty-four hours post-nuclefection, GFP positive cells were isolated by cell sorting and then cultured in μ-slide 8 well, glass bottom culture dishes for another 24 hours. The GFP fluorescence signals were captured by Nikon microscope equipped with Hamamatsu camera; and time-lapse imaging was performed via MetaMorph software. The intensity of GFP fluorescence was cell cycle dependent. At early time points, GFP fluorescence was detected in single cells (see FIG. 2A, 0 h, 7 h), then it disappeared during M and G1 phases (as detected by differential interference contrast imaging (see FIG. 2A, 8 h, 10 h, 12 h), and the gradually appeared in the two daughter cells during S phase (see FIG. 2A, 24 h). The cell cycle dependent expression of Cas9-GFP-Gemimin fusion protein is graphed in FIG. 2B. Thus, Cas9-GFP-Gemimin fusion protein is expressed and accumulates during duing S, G2, and early M phases of the cell cycle and is targeted for degradation during late mitosis or early G1 phase.

### Example 4

### Cas9-GFP-Geminin Increased HDR/NHEJ Ratio in U2OS Cells

Homologous recombination (HR) is generally restricted to the S and G2 phases of the cell cycle. Thus, double-strand breads (DSBs) introduced by a targeting endonuclease during the G1 phase are likely to be repaired via non-homologous end joining (NHEJ). Since Cas9-GFP-Gemimin fusion protein expression is limited to S/G2/M, DSBs introduced by this fusion should be repaired by homology directed repair (HDR), thereby increasing the HDR/NHEJ ratio.

To test this hypothesis, the activities of Cas9-GFP-Geminin fusion and Cas9 were compared at the AAVS1 locus in U2OS cells. The cells were transfected by Amaxa nuclefection with 4 μg of Cas9-GFP-Gemimin or Cas9 only plasmid DNA, along with 4 μg of AAVS1-sgRNA plasmid DNA and 300 pmol of AAVS1-ss oligodeoxynucleotide (ODN) per one million of cells. The target sequence of AAVS1-sgRNA is 5′-GGGCCACTAGGGACAGGATTGG-3′ (SEQ ID NO:23; PAM site is underlined). The AAVS1-ssODN sequence is

(SEQ ID NO: 24)
5′-GTTCTGGGTACTTTTATCTGTCCCCTCCACCCCACAGTGGGGCCACT
AGTGACAGGATTGGTGACAGAAAAGCCCCATCCTTAGGCCTCCTCCTTCC
TAG-3′.

(The target sequence of gRNA is underlined, a single mutant (G>T) was made to create a restriction enzyme site, and the SpeI restriction site is double-underlined.) Genomic DNAs were harvested 48 hours post-transfection, and the target region was amplified by PCR with the forward primer 5′-TTCGGGTCACCTCTCACTCC-3′ (SEQ ID NO: 25) and the reverse primer 5′-GGCTCCATCGTAAGCAAACC-3′ (SEQ ID NO:26). NHEJ was measured by Cel-1 assay and HDR was measure by RFLP assay.

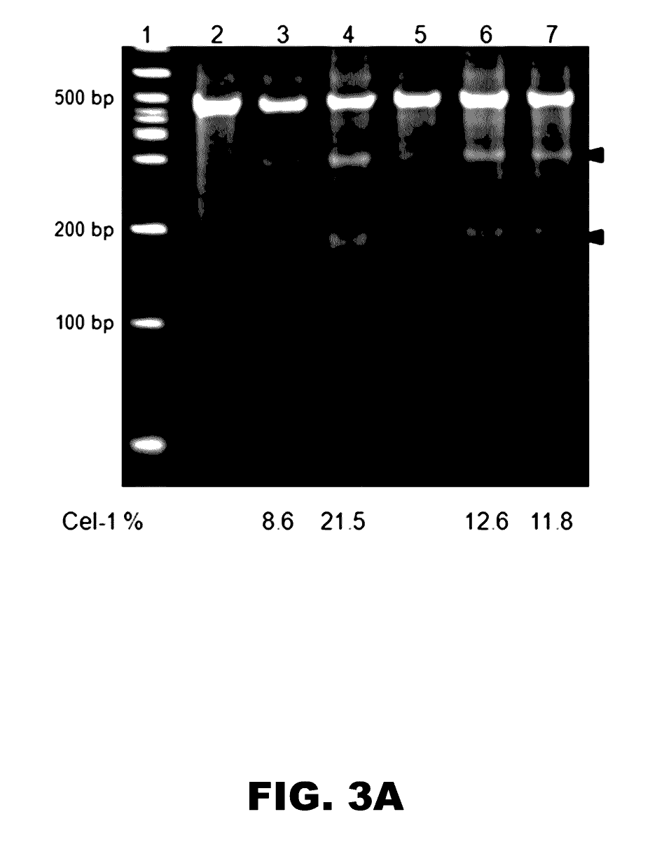

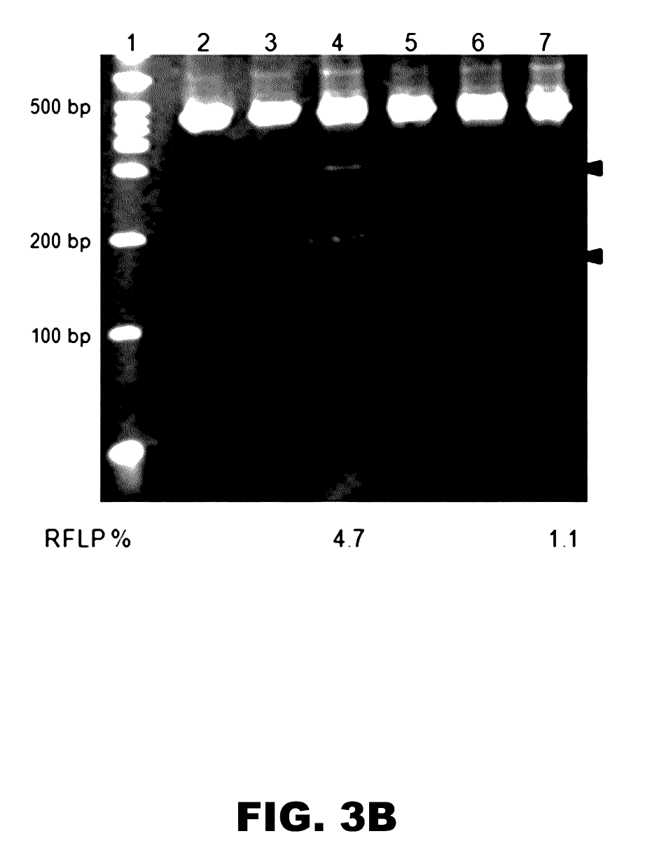

As shown in FIGS. 3A and 3B, Cas9-GFP-Geminin was able to achieve 4.7% HDR rate, with 8.6% of indels; while, Cas9 was only able to achieve 1.1° A HDR rate, with 12.6% of indels. These results indicated that Cas9-GFP-Geminin enhanced HDR/NHEJ ratio significantly in U2OS cells.

### Example 6

### Cas9-GFP-Geminin Increased HDR/NHEJ Ratio in K562 Cells

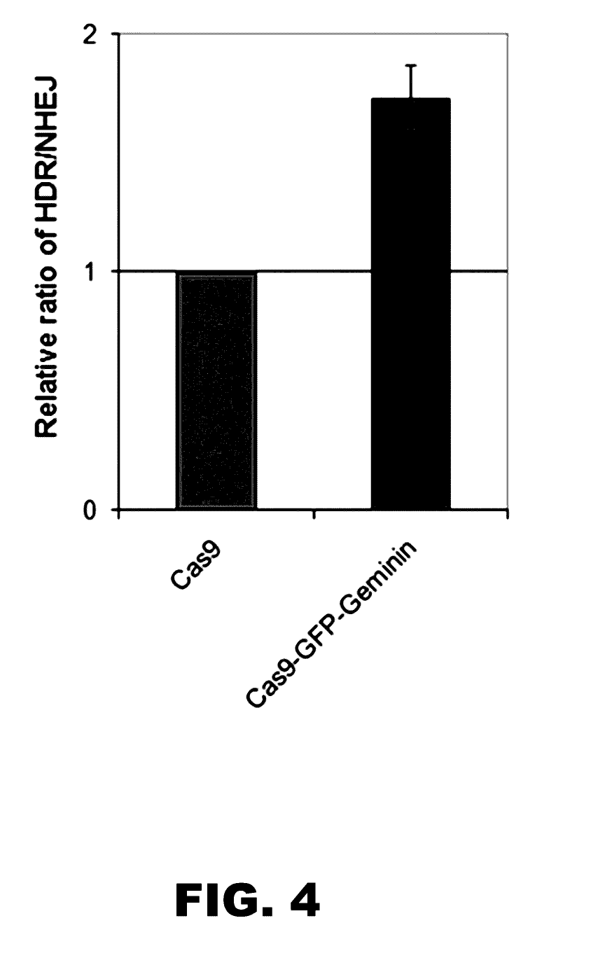

To test Cas9-GFP-Geminin's activity in other cell lines, K562 cells were transfected with Cas9-GFP-Gemimin or Cas9 plasmid DNA essentially as described above in Example 5. NHEJ and HDR were measured as described above. FIG. 4 presents the relative ratio of HDR to NHEJ from replicate samples. Cas9-GFP-Geminin increased the HDR/NHEJ ratio by about 1.7 fold in K562 cells (HDR/NHEJ ratio of Cas9 set to 1).

What is claimed is:

1. A fusion protein comprising a programmable DNA modification protein and a cell cycle regulated protein.

2. The fusion protein of claim 1, wherein the programmable DNA modification protein has nuclease activity, or the programmable DNA modification protein has non-nuclease activity.

3. The fusion protein of claim 2, wherein the programmable DNA modification protein having nuclease activity is chosen from a clustered regularly interspersed short palindromic repeats (CRISPR)/CRISPR-associated (Cas) (CRISPR/Cas) nuclease, a CRISPR/Cas nickase, a DNA-guided Argonaute endonuclease, a zinc finger nuclease, a transcription activator-like effector nuclease, a meganuclease, or a chimeric protein comprising a programmable DNA-binding domain and a nuclease domain.

4. The fusion protein of claim 3, wherein the CRISPR/Cas nuclease or nickase further comprises a guide RNA, and the DNA-guided Argonaute endonuclease further comprises a single-stranded guide DNA.

5. The fusion protein of claim 2, wherein the programmable DNA modification protein having non-nuclease activity is a chimeric protein comprising a programmable DNA-binding domain and a modification domain chosen from a transcriptional activation domain, a transcriptional repressor domain, a histone acetyltransferase domain, a histone deacetylase domain, a histone methyltransferase domain, a histone demethylase domain, a DNA methyltransferase domain, or a DNA demethylase domain.

6. The fusion protein of claim 5, wherein programmable DNA-binding domain is chosen from a CRISPR/Cas nuclease modified to lack all nuclease activity, a DNA-guided Argonaute endonuclease modified to lack all nuclease activity, a meganuclease modified to lack all nuclease activity, a zinc finger protein, or a transcription activator-like effector.

7. The fusion protein of claim 6, wherein CRISPR/Cas nuclease modified to lack all nuclease activity further comprises a guide RNA, and the DNA-guided Argonaute endonuclease modified to lack all nuclease activity further comprises single-stranded guide DNA.

8. The fusion protein of claim 1, wherein the cell cycle regulated protein is chosen from geminin, cyclin A, cyclin B, cyclin D, CDC20, or securin.

9. The fusion protein of claim 1, further comprising at least one nuclear localization signal, at least one cell-penetrating domain, at least one marker domain, and/or at least one linker.

10. The fusion protein of claim 1, wherein the programmable DNA modification protein is a Cas9 nuclease or derivative thereof and the cell cycle regulated protein is geminin.

11. The fusion protein of claim 1, which comprises SEQ ID NO:14.

12. A nucleic acid encoding the fusion protein of claim 1.

13. The nucleic acid of claim 12, which is operably linked to an expression control sequence.

14. The nucleic acid of claim 13, wherein the expression control sequence is a constitutive promoter sequence, a cell cycle regulated promoter sequence, a derivative, or fragment thereof.

15. The nucleic acid of claim 13, wherein the expression control sequence is a 3′ untranslated region that is targeted by one or more cell cycle regulated microRNAs, or the expression control sequence codes a reverse complement of a cell cycle regulated microRNA.

16. The nucleic acid of claim 12, which is codon optimized for translation in a eukaryotic cell.

17. The nucleic acid of claim 12, wherein the nucleic acid is part of a vector.

18. A cell comprising the nucleic acid of claim 12.

19. The cell of claim 18, wherein the nucleic acid is extrachromosomal, or the nucleic acid is integrated into a chromosome.

20. The cell of claim 18, wherein the fusion protein is degraded during M phase and/or during the transition from M phase to G1 phase.

21. The cell of claim 18, wherein the cell is a human cell, a non-human mammalian cell, a non-mammalian vertebrate cell, a stem cell, a non-human one cell embryo, an invertebrate cell, a plant cell, or a single cell eukaryotic organism.

22. A method for modifying a chromosomal sequence and/or regulating expression of a chromosomal sequence in a cell cycle dependent manner, the method comprising introducing into the cell a nucleic acid encoding the fusion protein comprising a programmable DNA modification protein and a cell cycle regulated protein, and optionally a donor polynucleotide comprising at least one sequence having substantial sequence identity with a target site in the chromosomal sequence, wherein the fusion protein is expressed during a portion of the cell cycle such that the fusion protein modifies the chromosomal sequence and/or regulates expression of the chromosomal sequence during that portion of the cell cycle.

23. The method of claim 22, wherein the programmable DNA modification protein of the fusion protein is chosen from a CRISPR/Cas nuclease system, a CRISPR/Cas nickase system, a DNA-guided Argonaute endonuclease system, a zinc finger nuclease, a transcription activator-like effector nuclease, a meganuclease, a chimeric protein comprising a programmable DNA-binding domain and a nuclease domain, or a chimeric protein comprising a programmable DNA-binding domain and a non-nuclease domain.

24. The method of claim 23, wherein the CRISPR/Cas nuclease system comprises a CRISPR/Cas nuclease and a guide RNA, the CRISPR/Cas nickase system comprises a CRISPR/Cas nickase and a pair of guide RNAs, and the DNA-guided Argonaute endonuclease system comprises an Argonaute endonuclease and a single-stranded guide DNA.

25. The method of claim 22, wherein the cell cycle regulated protein of the fusion protein is chosen from geminin, cyclin A, cyclin B, cyclin D, CDC20, or securin.

26. The method of claim 22, wherein the programmable DNA modification protein of the fusion protein is a targeting endonuclease that introduces a double-stranded break at a target site in the chromosomal sequence, and wherein repair of the double-stranded break has a ratio of homology directed repair (HDR) to non-homologous end joining (NHEJ) that is increased relative to a corresponding targeting endonuclease that is not fused to a cell cycle regulated protein.

27. The method of claim, wherein the cell is a human cell, a non-human mammalian cell, a non-mammalian vertebrate cell, a stem cell, a non-human one cell embryo, an invertebrate cell, a plant cell, or a single cell eukaryotic organism.


SEQUENCE LISTING (26 sequences)

SEQ ID NO: 1 - protein, 7 aa, Artificial Sequence
  SYNTHESIZED
PKKKRKV

SEQ ID NO: 2 - protein, 6 aa, Artificial Sequence
  SYNTHESIZED
KKKRRV

SEQ ID NO: 3 - protein, 16 aa, Artificial Sequence
  SYNTHESIZED
KRPAATKKAGQAKKKK

SEQ ID NO: 4 - protein, 20 aa, Artificial Sequence
  SYNTHESIZED
GRKKRRQRRRPPQPKKKRKV

SEQ ID NO: 5 - protein, 19 aa, Artificial Sequence
  SYNTHESIZED
PLSSIFSRIGDPPKKKRKV

SEQ ID NO: 6 - protein, 24 aa, Artificial Sequence
  SYNTHESIZED
GALFLGWLGAAGSTMGAPKKKRKV

SEQ ID NO: 7 - protein, 27 aa, Artificial Sequence
  SYNTHESIZED
GALFLGFLGAAGSTMGAWSQPKKKRKV

SEQ ID NO: 8 - protein, 21 aa, Artificial Sequence
  SYNTEHSIZED
KETWWETWWTEWSQPKKKRKV

SEQ ID NO: 9 - protein, 8 aa, Artificial Sequence
  SYNTHESIZED
GGSGGGSG

SEQ ID NO: 10 - protein, 5 aa, Artificial Sequence
  SYNTHESIZED
GGGGS

SEQ ID NO: 11 - protein, 5 aa, Artificial Sequence
  SYNTHESIZED
EAAAK

SEQ ID NO: 12 - protein, 7 aa, Artificial Sequence
  SYNTHESIZED
AEAAAKA

SEQ ID NO: 13 - protein, 5 aa, Artificial Sequence
  SYNTHESIZED
PAPAP

SEQ ID NO: 14 - DNA, 4101 bp, Streptococcus pyogenes
atggacaagaagtacagcatcggcctggacatcggcaccaactctgtgggctgggccgtgatcaccgacgactacaaggtgcccagcaagaaattcaaggtgctgggcaacaccgaccggcacagcatcaagaagaacctgatcggcgccctgctgttcggctctggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaagctggccgacagcaccgacaaggccgacctgagactgatctacctggccctggcccacatgatcaagttccggggccacttcctgatcgagggcgacctgaaccccgacaacagcgacgtggacaagctgttcatccagctggtgcagatctacaatcagctgttcgaggaaaaccccatcaacgccagcagagtggacgccaaggccatcctgagcgccagactgagcaagagcagacggctggaaaatctgatcgcccagctgcccggcgagaagcggaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacagcgagatcaccaaggcccccctgtccgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttcagaatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgcggaagcccgcctttctgagcggcgagcagaaaaaggccatcgtggacctgctgttcaagaccaaccggaaagtgaccgtgaagcagctgaaagaggactacttcaagaaaatcgagtgcttcgacagcgtggaaatcagcggcgtggaagatcggttcaacgcctccctgggcgcctatcacgatctgctgaaaattatcaaggacaaggacttcctggacaatgaggaaaacgaggacattctggaagatatcgtgctgaccctgacactgtttgaggaccggggcatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggacactctctgcacgagcagatcgccaatctggccggatcccccgccattaagaagggcatcctgcagacagtgaagattgtggacgagctcgtgaaagtgatgggccacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccacattgtgccccagtccttcatcaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcattaagcggcagctggtggaaacccggcagatcacaaagcacgtggcacagatcctggactcccggatgaacactaagtacgacgagaacgacaaactgatccgggaagtgaaagtgatcaccctgaagtccaagctggtgtccgacttcagaaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgattacaaggtgtacgacgtgcggaagatgatcgccaagagcgagcaggaaatcggcaaggctaccgccaagtacttcttctacagcaacatcatgaactttttcaagaccgagatcacactggccaacggcgagatcagaaagcggcctctgatcgagacaaacggcgaaaccggggagatcgtgtgggataagggccgggattttgccacagtgcggaaagtgctgtccatgccccaagtgaatatcgtgaaaaagaccgaggtgcagaccggcggcttcagcaaagagtctatcctgcccaagaggaactccgacaagctgatcgccagaaagaaggattgggaccctaagaagtacggcggctttgacagccccaccgtggcctactctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagcggatgctggcttctgccggcgaactgcagaagggaaacgagctggccctgccctccaaatatgtgaacttcctgtacctggccagccactatgagaagctgaagggctcccccgaggataatgagcagaaacagctgtttgtggaacagcacaagcactacctggacgagatcatcgagcagattagcgagttctccaagcgcgtgatcctggccgatgccaacctggacaaggtgctgagcgcctacaacaagcaccgggataagcccatcagagagcaggccgagaatatcatccacctgtttaccctgaccaacctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 15 - DNA, 21 bp, Artificial Sequence
  SYNTHESIZED
cccaagaaaaagcgcaaagtg

SEQ ID NO: 16 - DNA, 24 bp, Artificial Sequence
  SYNTHESIZED
ggcggctccggcggcggcagcggc

SEQ ID NO: 17 - DNA, 713 bp, Artificial Sequence
  SYNTHESIZED
agcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacagggc

SEQ ID NO: 18 - DNA, 330 bp, Homo sapiens
atgaatcccagtatgaagcagaaacaagaagaaatcaaagagaatataaagaatagttctgtcccaagaagaactctgaagatgattcagccttctgcatctggatctcttgttggaagagaaaatgagctgtccgcaggcttgtccaaaaggaaacatcggaatgaccacttaacatctacaacttccagccctggggttattgtcccagaatctagtgaaaataaaaatcttggaggagtcacccaggagtcatttgatcttatgattaaagaaaatccatcctctcagtattggaaggaagtggcagaaaaacggagaaaggcgctg

SEQ ID NO: 19 - protein, 1738 aa, Artificial Sequence
  SYNTHESIZED
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVGGSGGGSGSGGEELFAGIVPVLIELDGDVHGHKFSVRGEGEGDADYGKLEIKFICTTGKLPVPWPTLVTTLCYGIQCFARYPEHMKMNDFFKSAMPEGYIQERTIQFQDDGKYKTRGEVKFEGDTLVNRIELKGKDFKEDGNILGHKLEYSFNSHNVYIRPDKANNGLEANFKTRHNIEGGGVQLADHYQTNVPLGDGPVLIPINHYLSTQTKISKDRNEARDHMVLLESFSACCHTHGMDELYRAGGSGGGSGMNPSMKQKQEEIKENIKNSSVPRRTLKMIQPSASGSLVGRENELSAGLSKRKHRNDHLTSTTSSPGVIVPESSENKNLGGVTQESFDLMIKENPSSQYWKEVAEKRRKAL

SEQ ID NO: 20 - DNA, 21 bp, Artificial Sequence
  SYNTHESIZED
agttggagctggtggcgtagg

SEQ ID NO: 21 - DNA, 22 bp, Artificial Sequence
  SYNTHESIZED
ttatatccaacacttcgtgggg

SEQ ID NO: 22 - DNA, 9689 bp, Homo sapiens
gcagtctattcaccctcctcagtgtcatacctttctgctgtcttctgattgagttctctgcctacactctcctccaggtgatagttgtagcctttacagcaaaccagtggacaagaagcatcagggtctttggaaattttgctgtgcattggaccagtaaaagtaattccagatctgaagacagcttgactttggcttatttttactgattcctatttgtgtttttcagaaagagctacttgatcaccagctctagaagtatcaggagttacaattatccaatcttatgcaaattggctggtgggctgcaaagcttgtgtactttttgcagtgggggttgtacaaacagaaaaataaagaatacaagggtcgggccaggcacggtctctcatgcctgtaatcccagcactttgggaggtcgaggtgagaggatcacttgaaaccaggagttcgagaccagcatggccagcttggtgaaaccccgtctgtactaaaaatacaaaaattagctgggcatggtggcacacgcctgtagtcccagctactcgggaggctgagacaggagaattgcttgaacctgggaggtggaggttgcagtgagctgagattgtgccactgcactccagcctgggcgacagagtgagactgtctcaaaacaaaaaacaaggctcttctgaagacgctttaatgaaaatcattatttcttagtcaccccaagagcatgaatttgatgtggttgggaactcaagctaaatattgtgaaggtgtaactctgtgttgacctctagccatgcagctcagttgttttgcaaactgtcctgatttcccacagatgacttgtcctactgaggacacctatcagtaggtcagagagcagctttgtgagccttcctgctggtacccagaagtgagtttgtgcccactaattttttagcattttaattcctcgcaacagaagagactggcaaaactcaacaattctctgtatttatttatgtatttttgagacaaggtcttgccctatcacccaggctgatgtgcagtggcacgatcatggctcattgcagctttgacctcatgggtttaagggattctcccacctcagcctcctgagtagctgggaccacaggtgcaagccaccatgccctattaacttttttttttttttaagacagggttttgctgtctgtcacccaggctggagtacagtggtgcgatcttggctcactgcaacctccacctcctgggttcaaatgattctcctgtctcagctgaccgagtagctggtattacaggcatgtgccaccacacccagctaatttttgtatttttagtggagatggggtttaaccatgttggccaggctggtctcgaactcttgacctcaagtgttccacctgtcttggcctcccaaaatgttgggattacaggtgtgaactactgcacccagacaagaaaacacatacttatttttataaactataggaaagcacaaagaaaacaaaaatcatcgaaatctcattctccagataaaagcagctgacattttgctgcgacttgcaaaatgcctttggattcagataacagtggttctgaaactttagcgtgcatcagaattaactggagggcttgttaaaacagtgcttctgagtcagaagttttggagtggagccgataatttgaatttctttctttctttcttttttttttttttttgagacagtttccctcttgtttcccaggctggagtgcattggcacaatcttggctcactgcaacctccacctcctaggttcaagcaattcttctgcctcagcctctcgagtagctgggattacagatgcccgccaccatgcccagctaattttttgtatttctagtagagacagggtttcactgttggctacgctggtcttgaactcctgacctcaggcaatccacccatgtcagcctcctaaggtgctgggattacaggcatgagccaccacatccagctgataatttgaatttctaagaagctcccaggtgtccctgacactgttggtccaggtatcatacattgagaagcactggatatgtgcaccttggctgttccaagtagggtctgcaaccagaggcattgacatcattttgggaacttgtaatgcagaatctcaggccccagctcagacctactgaatcataatctgtaatttaataagatccctaaaaaatttttaagcaccaggcacggtggctcacgcgtgtaatcccagcactttgggaggccaagcgggtggatcacgaggtcaggagttcaagaccagcctggccaagatggtgaaaccctgtctctactaaaaatacaaaaattagccgggtgtggcggtgggcacctgtaatcccagctactcgggaggctgaggcagagaattgcttgaacctgggaggcagaggttgcagttagccgagatcgtgccactgtattccaacctaggtgacagagtgagactccatctcaaaaaaaaaaaaaaaaaaaatttttttaagcacaggtttgagaaggattggtttatattttaagcctcatagtatataacagttactccccccaccatattgaggtagaatttacacatagtgcaccattttataatgtataatttgatgagttttgacaaaatgatactaaatagttttgtacccttttgtctctctacccaacataatgaggactttcctgtagtattagatgttttggaaaaacatgacttctaatggctgtacaatacattgtaggtaaggatgttccagtttaaccaattcttcttttatttatttatttatttatttttgagacagagtctcttgctgttgcccagtctggactatagtggcgcagtcttggctcactgcaacctgcacttcctgggttcaagcgagtcttgtgtctcagcctcccaagtagctgagactacaggtgtgcaccaccacactcaggtaatttttgtattttcagtagagacagggtttcgacatgttgcccaggctggtctcctgagctcaggcaatctgcctgcctaggcctcccaaagtgctgggattacaggcgtgagccactgtacctggcccagtttaaccaattcttctattgtgagacatctatgttgttcccaatttctcaccagtgtaaataatgcttcaatgaatgcttttggacttaaatgttttcgtttggactttaacatatttttccacagctaaattactgaggaaagggtacgggacaggcaagaacaggtatccattactcaagaatgaaaagttaatgaattaaatttttctgtttgggtttcaggaaaaatggctagaaatcattaaaaaaaaaatccattgcagcagaaacagtgggatgcactgtatcttaaaaacaaaaagggccaggctgggcacagtggctcacgcctgtaatcccagcactttgggaggctgagatgggtggatcacctgaggtcaggaactcaagaccagcccggccaaactggtaaaactctgcctttactaaaaatacaaaaattagctgggtgtggtggcgtgcgcttgtaatcccaggtactcgggaggctgaggcaggagaatcgcttgaacctgggaggcggaggttgcagtgagccgaagctgtgccattccactccagcctgggcgacagaacgagactcaatcttaaaaaaaaaaaaaaaagaaaaaagccgggagtggtggcaggtgcctgcaatcctaggtacttgggaggctgaggcaggagaattgcttgagcccaggaggcggaggttgcagtgagctgaaatggtgccactgcactccagcctgggcagcagagcaagactctgtctcatggaaaaaataaaataaaaaaaaaaagactcagtaaacttactgttgaatcctttaccaattaatgcaacttttgagtcttttctcaatagccattcttttgtaattcataacttatatgtatttaaggaatgtttcatacacataggaaataaccacattctataaagggtctaaatacataaaactatcacgtttattagcaaatctttatatcctttaatgtgtcagtagcttaagaaataatgaaggccgaaggccaggcgcagtggctcacgcctgtaatcccagcactttgggaggccgaggcgggtggatcacgaggtcaggagatcgagaccatcatggctaacatggtgaaaccctgtctctactaaaaatataaaaaattagccaggcgtggtggcaggcggctgtagtcccagctacttgggaggctgaggcaggagaatcgcttgaacctgggaggcggaggttgcagtgagctgagattgtgccactgcactccagcctgggcggcagagtcagattccatttcaaaaaaaaaataaataaataaaagaaaaaaaaaagaaataatgaataggcctggcatggtggctcacgcctgtaatcgcagctctttgggaggttgaggcaggtggatcacttgagcccaggagttccagaacagccggggcaacatagtgagaccctgcctctacaaaaaatacaaaaattagccaggtgtggtggtgtgtacctgtggtcccagctatttgggaggctgaggcaggaggatcgcttgagcccaggaggcagaggttgcagtgggccgagattgagccactgcactccagcctggatggtagagtgaaaccttgtctcaaaaaaagaaaaaaagaaaaaaaagagtcaaggaaacattatccgctttcagttagcaaggtctttactcatcaggaaatgtaaaacttctactttcaaaagagaactattggccgggcgcggtggctcaggcctgtaatcccagcactttgggacgcggaggcaggcggattgcctgagctcagaccagcctgggcaacatggtgaaaccccatctctactaaaaatacaaaaaatttaagctgggcgtggtggctcatgcctgtaatcccagcactttgggtgtctgaagtgggacgatcacttgaggtcaggaattcgagaccagcctggacaacatggtgaaactccatctctactaaaaatacaaaaattaactgtaatttttgtattccctgtgatcccagccacttgggaggctgaggcatgagaatcacttgaaccaggcaggcggaggttatagtgagccgagatcgtgccactgcactccagcctgggtgatagagcaagacaagactttatcccccaaaaaacaaaaaaacccagaaaatcccacaaataaaaacacaaagaattagccaggcatggcagtaggcgcctgtagtcccagctacttgggaggctgaggcatgagaattgcttgaccttgggaggcagaaagcagagaattgcagtgagctgagatcgtaccactgcactccagcctgggtgccaaaatgagattctatctccaaaaaaaaaaaaaaggaaaaatatttgattcttttactttctaaaaagagtttacatactttcctcccactatttattttgtaaacaactggcatatttaccagatggggatttcatctttgatttgtaatctgcttttttccacttggcaatgtcgtgaacatctatcttttcatgtcaataaatgtcaataaataaacagtatagatgatcattcatttttttttttttttgagacagtcttgctctgttgcccaggctggagtgcagtgccatcatggctcactgcagccccctgggctcaagcaatactcctgcctcagccttccaagtagctgggaccacaggcatgcaccaccatgtccagctgatttttacctttttttttgtagagatgggggtctcactacgttgcccaggctggtctcaaactcctgggctcaagcaatcttcccacttcagcctcccaaagtgctgggaatacatgtatgaaccactgtgcctggtctacctgatcattttttttttcttgatggaatttcactcatgttacccaggatggagtgcaatagcacgatcttggctcactgcaacctccacctcctgggttcaagcgattctcctgcctcagcctcctgagtagttgggattacaggtgcacgccaccacacctggctaatttttgtatttttagtagagacggggtttcaccatgttggtcaggctggtctcgaactcctgacctcgtggtctgcttgccttgggctcccaaagtgctgggattacaggcgtgagccactgcgcctggcctacatgatcattcctaataggcacctggtattccatatttaccattttaaccttttggacatttaggttattttccattttattattacagcaacttcaataagcatctttgcatgtggctttgttttgatatagttgtacattcacatagttttaagaaatggatcaggccgggcatggtggctcacgcctgtaatcccagcactttgggaggctgaggtgggcggatcacaaggtcaggagtttgagaccagccgggccaacatggtgaaaccctgtctctactaaaaatacaaaaattagctgggcgtggtggcatgcacctataatgccagctactcgggaggctgaggcaggagaatcgtttgtacccgggaggcagaagttgcaatgagtcaagatggccccagtgcactccagcctgggcgacagagcaagactctgtcccagaaaaaaaaaaaaagaaatggatcagaaacaaggactctttctgaaaggaaaaaaaaaagaatggagatccatcgtatactttgcccatttcccaattttgcaaaattatatagtaaccagaatacttacattgaagcaacccattgatcttactcagatttacttatactcatatttgtgtgtgtttacatagttttttgcatgtctgattcttctgtcaaacgaaattcctttttttttttttttttgagacagggacttgctcaggctggaatgcagtggcacaatctctggtcactgtaacctctgcttcctgggctcaagcaatcttccctccttggcctcccaaactgctgggattacaggtgtgagccaccatgcctggcccagatttctttgaaagggctaattcctccatatctttgtcaacactacttttgggttttgttcagtttatccctctgtaactcaagattactttttttatagttactttttaaatagtttttgacatttaaatatttcatctatttgaacttaattttggtgtaaggtgtgaaagagatttatctgattttttttctaaatggattagccagttgcctcaatatatcttactgataccatcaagtagttgactaggttatcaaaatagttgttaaaggaaggtatcattaaaaaaaaaagatacatgcatatttactgatcaagtgtggtggagatgaagaacttagtcctcatgtataaaatctcaataaagagtctttggccttaattaggtcttaatgcctatctcttggacttatcaccttagccagaggctgtaaggtctgtcacaatatgattggaatgcttctgaaagggaagtgaagactatattttagaataaggaaaagggtgtagtgtgtgttttaaaagaggcattctatgggttgcaatgtttagaacattttattaaagtacaaaattgttggaatttagctaatagaaaaacatagtaaatatttacaaaaacgttgataacattactcaagtcacacacatataacaatgtagacaggtcttaacaaagtttacaaattgaaattatggagatttcccaaaatgaatctaatagctcattgctgagcatggttatcaatataacatttaagatcttggatcaaatgttgtccccgagtcttctgcaatccagtcctcttagaaattggtttctctctttgggagattcagactcagaggcagccagaggggacaggtcaagagctgaaataatcacataactactctaattttcttcattctattgactgtgtcaagttatagacacagccaaagtgtttttcttcggcctctgatgatttgagaagatgaagaacatgagcaatttctcattgcttaaagaaaaacttggcacataagaggctgagtgtagtagagtatctgtactagaaccataaagttctatctgatggtaaattatgtataaaactaagataaaacagataattatgctctatctcatatctactgaaagtagaaaaggaggaagagtgacacttttaaatcaaactgctctagttttagcttagtggatggttaataaacacactgctttacgctgaagtgatcagatagctatttctacagttcagaagaacttaaaaatcaggttttaaagacaaaagaaagcagactcaaaacacagacaaagcagagaagaaaacaatgcccatgagatggtcactatttagacagtattataaaaagctaaagaacacttgggctttacttcactttgatgtcttgtactaaaaacaccttccccaaactaaattcagaggggaggaagttaagagcttcaggtaactttaaaaccagtcttgggcttggtaagataattacttaaaataatcgcctcacattttaaaacagatcatcttcatctgactcttccaggtactttataggtttctttgcccgtacagattttgcccgaggagccacagctgagtcaaagtccatatggaagtcatcactctcccccttggatttctaaaagagaaaagcccaggtaacttgcacattgtaaatctgacaacataattgtaatgtaaaaaaatgtatcaagacactatattcaaggagttttctattttctaccaagtaataagaagcagatctaaggccaactcttccattgcccaaataagtggcatatttaactttgttaaaactaaatatgtacagtaaaagctaacagaatatgagagttaattttcttaaagatatgccaaatttttaagagcaatggcttagttacgtgtttcagaacatctacagcaaaaggactgactaggatcaacactcaccttgcttgtgactgctttcgaaacaattttctcaaaattagagtcagaatcatcagaagtggatggcttccttttgcggcgattcttggttttggcaggatcaggcttttgagagacaccagaattcaaagctggatcccttttagttccttttggggcagccctttttttggcaccggtagtggaggtggaagactgacctgcaattcaatacaggcatttgtcacagctgctctttttttgagatggggtctcactctatcgtccaggctggagtgcagtggtgttatctcggctcactgcaacctctgcctcctgggttcaagcgattctcctgcctcagcctcctgagtagctgggattacaggcgtgtgccaccacacccggctaattttttgtatttttagtagagatgggattccaccatgttggtcaagctggtctcaaactcctgacctcaggtgatccactcgcctcggcctcccaaagtgctgggattacaggcatgagcaaccgcgcctgacctagtcacagccactcttagatgaattgttctcattgcgaactttcttcagcaatgtgatg

SEQ ID NO: 23 - DNA, 22 bp, Homo sapiens
gggccactagggacaggattgg

SEQ ID NO: 24 - DNA, 100 bp, Artificial Sequence
  SYNTHESIZED
gttctgggtacttttatctgtcccctccaccccacagtggggccactagtgacaggattggtgacagaaaagccccatccttaggcctcctccttcctag

SEQ ID NO: 25 - DNA, 20 bp, Artificial Sequence
  SYNTHESIZED
ttcgggtcacctctcactcc

SEQ ID NO: 26 - DNA, 20 bp, Artificial Sequence
  SYNTHESIZED
ggctccatcgtaagcaaacc